SEQUENCE LISTING (66 sequences)

SEQ ID NO: 1 - protein, 463 aa, Artificial sequence
  Modified Homo sapiens immunoglobulin G1 heavy chain
MNLLLILTFVAAAVAQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSV

SEQ ID NO: 3 - protein, 234 aa, Artificial sequence
  Modified Homo sapiens immunoglobulin G1 kappa light chain
MNLLLILTFVAAAVADVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 113 aa, Mus musculus
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKR

SEQ ID NO: 5 - protein, 327 aa, Equus caballus
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIVIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 6 - protein, 415 aa, Equus caballus
SLEDTAVIPLFSECKAPKEDDVVSLACLVKGYFPEPVQVTWEPEMQNQKPWTFPAMKKGQEYIHVFSLTTWWKPGSHSCTVHHKASSFRKKMTFQEPASWAPQRTSALPVTSKEPTPAPTTLRKSEPSTRHTQPETQKPRIPVDTPLKECQSHTHPPSIYLLHPPLQGLWLKGEATFTCLVVGDDLKDAHLSWELSERSNGMFVESGPLEKHTNGSQSRSSRLALPRSSWAMGTSVTCKLSYPNLLSSMEVVGLKEHAASAPRSLTVHALTTPGLNASPGATSWLQCKVSGFSPPEIVLTWLEGQREVDPSWFATARPTAQPGNTTFQTWSILLVPTIPGPPTATYTCVVGHEASRQLLNTSWSLDTGGLAMTPESKDENSDDYADLDDAGSLWLTFMALFLITLLYSGFVTFIK

SEQ ID NO: 7 - protein, 334 aa, Canis familiaris
SKTSPSVFPLSLCHQESEGYVVIGCLVQGFFPPEPVNVTWNAGKDSTSVKNFPPMKAATGSLYTMSSQLTLPAAQCPDDSSVKCQVQHASSPSKAVSVPCKDNSHPCHPCPSCNEPRLSLQKPALEDLLLGSNASLTCTLSGLKDPKGATFTWNPSKGKEPIQKNPERDSCGCYSVSSVLPGCADPWNHGDTFSCTATHPESKSPITVSITKTTEHIPPQVHLLPPPSEELALNELVTLTCLVRGFKPKDVLVRWLQGTQELPQEKYLTWEPLKEPDQTNMFAVTSMLRVTAEDWKQGEKFSCMVGHEALPMSFTQKTIDRLAGKPTHVNVSVV

SEQ ID NO: 8 - protein, 426 aa, Canis familiaris
TSQDLSVFPLASCCKDNIASTSVTLGCLVTGYLPMSTTVTWDTGSLNKNVTTFPTTFHETYGLHSIVSQVTASGKWAKQRFTCSVAHAESTAINKTFSACALNFIPPTVKLFHSSCNPVGDTHTTIQLLCLISGYVPGDMEVIWLVDGQKATNIFPYTAPGTKEGNVTSTHSELNITQGEWVSQKTYTCQVTYQGFTFKDEARKCSESDPRGVTSYLSPPSPLDLYVHKAPKITCLVVDLATMEGMNLTWYRESKEPVNPGPLNKKDHFNGTITVTSTLPVNTNDWIEGETYYCRVTHPHLPKDIVRSIAKAPGKRAPPDVYLFLPPEEEQGTKDRVTLTCLIQNFFPADISVQWLRNDSPIQTDQYTTTGPHKVSGSRPAFFIFSRLEVSRVDWEQKNKFTCQVVHEALSGSRILQKWVSKTPGK

SEQ ID NO: 9 - protein, 335 aa, Felis catus
ASTTASSVFPLAPSCGTTSGATVALACLVLGYFPEPVTVSWNSGALTSGVHTFPSVLQASGLYSLSSMVTVPSSRWLSDTFTCNVAHRPSSTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 10 - protein, 96 aa, Camelus dromedarius
EVQLVESGGGLVQPGGSLRLSCAASGFTFSSYDMSWVRQAPGREREGVAAINSGGGSTYYADSVKGRFTISQDNAKNTVYLQMNSLKPEDTAMYYC

SEQ ID NO: 11 - protein, 96 aa, Camelus dromedarius
EVQLVESGGGLVQPGGSLRLSCAASGFTFSSYWMYWVRQAPGKGLEWVSTINSGGGSTYYADSVKGRFTISRDNAKNMLYLQMNSLKPEDTAMYYC

SEQ ID NO: 12 - DNA, 1434 bp, Artificial sequence
  Murine anti-AGE IgG2b heavy chain
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctaagaccacccctccctccgtgtaccctctggctcctggctgtggcgacaccaccggatcctctgtgaccctgggctgcctcgtgaagggctacttccctgagtccgtgaccgtgacctggaactccggctccctgtcctcctccgtgcacacctttccagccctgctgcagtccggcctgtacaccatgtcctccagcgtgacagtgccctcctccacctggccttcccagaccgtgacatgctctgtggcccaccctgcctcttccaccaccgtggacaagaagctggaaccctccggccccatctccaccatcaacccttgccctccctgcaaagaatgccacaagtgccctgcccccaacctggaaggcggcccttccgtgttcatcttcccacccaacatcaaggacgtgctgatgatctccctgacccccaaagtgacctgcgtggtggtggacgtgtccgaggacgaccctgacgtgcagatcagttggttcgtgaacaacgtggaagtgcacaccgcccagacccagacacacagagaggactacaacagcaccatcagagtggtgtctaccctgcccatccagcaccaggactggatgtccggcaaagaattcaagtgcaaagtgaacaacaaggacctgcccagccccatcgagcggaccatctccaagatcaagggcctcgtgcgggctccccaggtgtacattctgcctccaccagccgagcagctgtcccggaaggatgtgtctctgacatgtctggtcgtgggcttcaaccccggcgacatctccgtggaatggacctccaacggccacaccgaggaaaactacaaggacaccgcccctgtgctggactccgacggctcctacttcatctactccaagctgaacatgaagacctccaagtgggaaaagaccgactccttctcctgcaacgtgcggcacgagggcctgaagaactactacctgaagaaaaccatctcccggtcccccggctag

SEQ ID NO: 13 - DNA, 1416 bp, Artificial sequence
  Chimeric anti-AGE human IgG1 antibody heavy chain
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 14 - DNA, 720 bp, Artificial sequence
  Murine anti-AGE Kappa light chain
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 15 - DNA, 720 bp, Artificial sequence
  Chimeric anti-AGE human kappa light chain
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 16 - protein, 477 aa, Artificial sequence
  Murine anti-AGE IgG2b heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 17 - protein, 471 aa, Artificial sequence
  Chimeric anti-AGE human IgG1 heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 239 aa, Artificial sequence
  Murine anti-AGE kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 239 aa, Artificial sequence
  Chimeric anti-AGE human kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 118 aa, Artificial sequence; identical to SEQ ID NO: 2
  Murine anti-AGE IgG2b heavy chain (variable region)

SEQ ID NO: 21 - protein, 112 aa, Artificial sequence
  Murine anti-AGE kappa light chain (variable region)
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIK

SEQ ID NO: 22 - protein, 326 aa, Artificial sequence
  Human constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 23 - protein, 7 aa, Artificial sequence
  CDR1H (heavy chain)
SYTMGVS

SEQ ID NO: 24 - protein, 17 aa, Artificial sequence
  CDR2H (heavy chain)
TISSGGGSTYYPDSVKG

SEQ ID NO: 25 - protein, 10 aa, Artificial sequence
  CDR3H (heavy chain)
  misc_feature (10)..(10): Xaa can be any naturally occurring amino acid
QGGWLPPFAX

SEQ ID NO: 26 - protein, 17 aa, Artificial sequence
  CDR1L (light chain)
RASKSVSTSSRGYSYMH

SEQ ID NO: 27 - protein, 7 aa, Artificial sequence
  CDR2L (light chain)
LVSNLES

SEQ ID NO: 28 - protein, 9 aa, Artificial sequence
  CDR3L (light chain)
QHIRELTRS

SEQ ID NO: 29 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNQKFQGRVTMTVDKSTNTVYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaacagaaattccagggcagagtgaccatgaccgtggacaagtccaccaacaccgtgtacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 31 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSTNTAYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccaccaacaccgcttacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 33 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSINTAYMELSRLRSDDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccatcaacaccgcttacatggaactgtccagactgcggagcgatgacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 35 - protein, 238 aa, Artificial Sequence
  Humanized light chain
METDTLLLWVLLLWVPGSTGDVVMTQSPLSLPVTLGQPASISCRSSQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 715 bp, Artificial Sequence
  Humanized light chain
atggagaccgacaccctgctgctctgggtgctgctgctctgggtgcccggctccaccggagacgtcgtgatgacccagtcccctctgtccctgcctgtgaccctgggacagcctgcctccatctcctcagatcctcccagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 37 - protein, 238 aa, Artificial Sequence
  Humanized light chain
METDTLLLWVLLLWVPGSTGDVVMTQSPLSLPVTLGQPASISCRSRQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 715 bp, Artificial Sequence
  Humanized light chain
atggagaccgacaccctgctgctctgggtgctgctgctctgggtgcccggctccaccggagacgtcgtgatgacccagtcccctctgtccctgcctgtgaccctgggacagcctgcctccatctcctcagatccaggcagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 39 - protein, 238 aa, Artificial Sequence
  Humanized light chain
METDTLLLWVLLLWVPGSTGDVVMTQSPLSSPVTLGQPASISCRSSQSLVNSNGNTFLQWYHQRPGQPPRLLIYKVSLRFSGVPDRFSGSGAGKDFTLKISRVEAEDVGVYYCSQSTHVPPTFGQGTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 715 bp, Artificial Sequence
  Humanized light chain
atggagaccgacaccctgctgctctgggtgctgctgctctgggtgcccggctccaccggagacgtcgtgatgacccagtcccctctgtccagtcctgtgaccctgggacagcctgcctccatctcctcagatcctcccagtccctcgtgaactccaacggcaacaccttcctgcagtggtatcaccagcggcctggccagcctcccagactgctgatctacaaggtgtccctgcggttctccggcgtgcccgacgattttccggctctggcgctggcaaggacttcaccctgaagatctcccgggtggaagccgaggacgtgggcgtgtactactgctcccagagcacccacgtgccccctacatttggccagggcaccaactggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 41 - protein, 5 aa, Mus musculus
TYWMH

SEQ ID NO: 42 - protein, 17 aa, Mus musculus
EISPTNGRAYYNARFKS

SEQ ID NO: 43 - protein, 9 aa, Mus musculus
AYGNYEFAY

SEQ ID NO: 44 - protein, 16 aa, Mus musculus
RSRQSLVNSNGNTFLQ

SEQ ID NO: 45 - protein, 7 aa, Mus musculus
KVSLRFS

SEQ ID NO: 46 - protein, 9 aa, Mus musculus
SQSTHVPPT

SEQ ID NO: 47 - protein, 467 aa, Mus musculus
MGWTLVFLFLLSVTAGVHSQVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 467 aa, Artificial sequence
  Humanized heavy chain
MGWTLVFLFLLSVTAGVHSEVQLLESGAEAKKPGASVKLSCKASGYLFTTYWMHWVHQAPGQRLEWMGEISPTNGRAYYNARFKSRVTITVDKSASTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 467 aa, Artificial sequence
  Humanized heavy chain
MGWTLVFLFLLSVTAGVHSQVQLVQSGAEVKKPGASVKVSCKASGYLFTTYWMHWVRQAPGQRLEWIGEISPTNGRAYYNARFKSRVTITRDTSASTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 467 aa, Artificial sequence
  Humanized heavy chain
MGWTLVFLFLLSVTAGVHSQVQLVQSGAEVKKPGSSVKVSCKASGYLFTTYWMHWVRQAPGQGLEWMGEISPTNGRAYYNARFKSRVTITADKSTSTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 467 aa, Artificial sequence
  Humanized heavy chain
MGWTLVFLFLLSVTAGVHSQVQLVQSGAEVKKPGASVKVSCEASGYLFTTYWMHWVRQAPGQGLEWMGEISPTNGRAYYNARFKSRVTITRDTSINTAYMELSRLRSDDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSV

SEQ ID NO: 53 - protein, 118 aa, Artificial sequence
  Humanized heavy chain variable region
EVQLLESGAEAKKPGASVKLSCKASGYLFTTYWMHWVHQAPGQRLEWMGEISPTNGRAYYNARFKSRVTITVDKSASTAYMELSSLRSEDTAVYYCARSFGNYEFAYWGQGTLVTVSS

SEQ ID NO: 54 - protein, 118 aa, Artificial sequence
  Humanized heavy chain variable region
QVQLVQSGAEVKKPGASVKVSCKASGYLFTTYWMHWVRQAPGQRLEWIGEISPTNGRAYYNARFKSRVTITRDTSASTAYMELSSLRSEDTAVYYCARSFGNYEFAYWGQGTLVTVSS

SEQ ID NO: 55 - protein, 118 aa, Artificial sequence
  Humanized heavy chain variable region
QVQLVQSGAEVKKPGSSVKVSCKASGYLFTTYWMHWVRQAPGQGLEWMGEISPTNGRAYYNARFKSRVTITADKSTSTAYMELSSLRSEDTAVYYCARSFGNYEFAYWGQGTLVTVSS

SEQ ID NO: 56 - protein, 118 aa, Artificial sequence
  Humanized heavy chain variable region
QVQLVQSGAEVKKPGASVKVSCEASGYLFTTYWMHWVRQAPGQGLEWMGEISPTNGRAYYNARFKSRVTITRDTSINTAYMELSRLRSDDTAVYYCARSFGNYEFAYWGQGTLVTVSS

SEQ ID NO: 57 - protein, 239 aa, Mus musculus
MVSSAQFLGLLLLCFQGTRCDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 239 aa, Artificial sequence
  Humanized light chain
MVSSAQFLGLLLLCFQGTRCDIVMTQTPLSLPVTLGQPASISCRSRQSLVNSNGNTFLQWLQQRPGQPPRLLIYKVSLRFSGVPDRFSGSGAGTDFTLTISRVEAEDVGIYFCSQSTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 239 aa, Artificial sequence
  Humanized light chain
MVSSAQFLGLLLLCFQGTRCDIVMTQTPLSLSVTPGQPASISCRSRQSLVNSNGNTFLQWYLQKPGQSPQLLIYKVSLRFSGVPDRFSGSGSGTDFTLKISRVEPEDVGVYYCSQSTHVPPTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 239 aa, Artificial sequence
  Humanized light chain
MVSSAQFLGLLLLCFQGTRCDVVMTQSPLSLPVTLGQPASISCRSRQSLVNSNGNTFLQWFQQRPGQSPRRLIYKVSLRFSGVPDRFSGSGSDTDFTLRISRVEAEDVGLYYCSQSTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 239 aa, Artificial sequence
  Humanized light chain
MVSSAQFLGLLLLCFQGTRCDIVMTQTPLSLSVTPGQPASISCRSRQSLVNSNGNTFLQWLLQKPGQPPQLLIYKVSLRFSGVPNRFSGSGSGTDFTLKISRVEAEDVGLYYCSQSTHVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 112 aa, Mus musculus; identical to SEQ ID NO: 21

SEQ ID NO: 63 - protein, 112 aa, Artificial sequence
  Humanized light chain variable region
DIVMTQTPLSLPVTLGQPASISCRSRQSLVNSNGNTFLQWLQQRPGQPPRLLIYKVSLRFSGVPDRFSGSGAGTDFTLTISRVEAEDVGIYFCSQSTHVPPTFGQGTKVEIK

SEQ ID NO: 64 - protein, 112 aa, Artificial sequence
  Humanized light chain variable region
DIVMTQTPLSLSVTPGQPASISCRSRQSLVNSNGNTFLQWYLQKPGQSPQLLIYKVSLRFSGVPDRFSGSGSGTDFTLKISRVEPEDVGVYYCSQSTHVPPTFGGGTKVEVK

SEQ ID NO: 65 - protein, 112 aa, Artificial sequence
  Humanized light chain variable region
DVVMTQSPLSLPVTLGQPASISCRSRQSLVNSNGNTFLQWFQQRPGQSPRRLIYKVSLRFSGVPDRFSGSGSDTDFTLRISRVEAEDVGLYYCSQSTHVPPTFGQGTKLEIK

SEQ ID NO: 66 - protein, 112 aa, Artificial sequence
  Humanized light chain variable region
DIVMTQTPLSLSVTPGQPASISCRSRQSLVNSNGNTFLQWLLQKPGQPPQLLIYKVSLRFSGVPNRFSGSGSGTDFTLKISRVEAEDVGLYYCSQSTHVPPTFGGGTKVEIK

(19) United States
(12) Patent Application Publication
(10) Pub. No.: US 2021/0253739 A1
(43) Pub. Date: Aug. 19, 2021

(54) ANTICARBOXYMETHYL LYSINE ANTIBODIES AND ULTRASOUND FOR REMOVING AGE-MODIFIED CELLS

(71) Applicant: SIWA Corporation, Chicago, IL (US)

(72) Inventor: Lewis S. Gruber, Chicago, IL (US)

(21) Appl. No.: 17/268,413

(22) PCT Filed: Aug. 22, 2019
(86) PCT No.: PCT/US2019/047762

§ 371 Date: Feb. 12, 2021

Related U.S. Application Data
(60) Provisional application No. 62/721,932, filed on Aug. 23, 2018.

(51) Int. Cl. C07K 16/44; A61K 47/69; A61K 47/68; A61P 35/04; A61P 19/02; A61N 7/00

(52) U.S. Cl. CPC C07K 16/44; A61K 47/6925; A61K 47/6843; A61K 2039/505; A61P 19/02; A61N 7/00; C07K 2317/92; A61P 35/04

(57) ABSTRACT

A method of killing AGE-modified cells comprises applying ultrasound to a subject and administering to the subject a composition comprising an anti-AGE antibody. The applying of ultrasound may occur before or after the administering of the anti-AGE antibody. The AGE-modified cells may be in a restricted site.

### SEQUENCE LISTING

The sequence listing contained in the ASCII text file named “SIW01-028-WO_Sequence_Listing.txt”, created on Jul. 10, 2019, with a file size of 120 kilobytes, is herein incorporated by reference.

### BACKGROUND

Senescent cells are cells that are partially-functional or non-functional and are in a state of proliferative arrest. Senescence is a distinct state of a cell, and is associated with biomarkers, such as activation of the biomarker p16^{Ink4a}, and expression of β-galactosidase. Replicative senescence results from telomere shortening that leads to DNA damage response. Senescence may also be caused by damage or stress (such as overstimulation by growth factors) of cells.

Advanced glycation end-products (AGEs; also referred to as AGE-modified proteins, or glycation end-products) arise from a non-enzymatic reaction of sugars with protein side-chains (Ando, K. et al., Membrane Proteins of Human Erythrocytes Are Modified by Advanced Glycation End Products during Aging in the Circulation, Biochem Biophys Res Commun., Vol. 258, 123, 125 (1999)). This process begins with a reversible reaction between the reducing sugar and the amino group to form a Schiff base, which proceeds to form a covalently-bonded Amadori rearrangement product. Once formed, the Amadori product undergoes further rearrangement to produce AGEs. Hyperglycemia, caused by diabetes mellitus (DM), and oxidative stress promote this post-translational modification of membrane proteins (Lindsey J B, et al., “Receptor For Advanced Glycation End-Products (RAGE) and soluble RAGE (sRAGE): Cardiovascular Implications,” Diabetes Vascular Disease Research, Vol. 6(1), 7-14, (2009)). AGEs may also be formed from other processes. For example, the advanced glycation end product, N^{ε}-(carboxymethyl)lysine, is a product of both lipid peroxidation and glycoxidation reactions. AGEs have been associated with several pathological conditions including diabetic complications, inflammation, retinopathy, nephropathy, atherosclerosis, stroke, endothelial cell dysfunction, and neurodegenerative disorders (Bierhaus A, “AGEs and their interaction with AGE-receptors in vascular disease and diabetes mellitus. I. The AGE concept,” Cardiovasc Res, Vol. 37(3), 586-600 (1998)).

AGE-modified proteins are also a marker of senescent cells. This association between glycation end-product and senescence is well known in the art. See, for example, Gruber, L. (WO 2009/143411, 26 Nov. 2009), Ando, K. et al. (Membrane Proteins of Human Erythrocytes Are Modified by Advanced Glycation End Products during Aging in the Circulation, Biochem Biophys Res Commun., Vol. 258, 123, 125 (1999)), Ahmed, E. K. et al. (“Protein Modification and Replicative Senescence of WI-38 Human Embryonic Fibroblasts” Aging Cells, vol. 9, 252, 260 (2010)), Vlassara, H. et al. (Advanced Glycosylation Endproducts on Erythrocyte Cell Surface Induce Receptor-Mediated Phagocytosis by Macrophages, J. Exp. Med., Vol. 166, 539, 545 (1987)) and Vlassara et al. (“High-affinity-receptor-mediated Uptake and Degradation of Glucose-modified Proteins: A Potential Mechanism for the Removal of Senescent Macromolecules” Proc. Natl. Acad. Sci. USAI, Vol. 82, 5588, 5591 (1985)). Furthermore, Ahmed, E. K. et al. indicates that glycation end-products are “one of the major causes of spontaneous damage to cellular and extracellular proteins” (Ahmed, E. K. et al., see above, page 353). Accordingly, the accumulation of glycation end-products is associated with senescence and lack of function.

The damage or stress that causes cellular senescence also negatively impacts mitochondrial DNA in the cells to cause them to produce free radicals which react with sugars in the cell to form methyl glyoxal (MG). MG in turn reacts with proteins or lipids to generate advanced glycation end products. In the case of the protein component lysine, MG reacts to form carboxymethyllysine, which is an AGE.

Damage or stress to mitochondrial DNA also sets off a DNA damage response which induces the cell to produce cell cycle blocking proteins. These blocking proteins prevent the cell from dividing. Continued damage or stress causes mTOR production, which in turn activates protein synthesis and inactivates protein breakdown. Further stimulation of the cells leads to programmed cell death (apoptosis).

p16 is a protein involved in regulation of the cell cycle, by inhibiting the S phase (synthesis phase). It can be activated during ageing or in response to various stresses, such as DNA damage, oxidative stress or exposure to drugs. p16 is typically considered a tumor suppressor protein, causing a cell to become senescent in response to DNA damage and irreversibly preventing the cell from entering a hyperproliferative state. However, there has been some ambiguity in this regard, as some tumors show overexpression of p16, while other show downregulated expression. Evidence suggests that overexpression of p16 is some tumors results from a defective retinoblastoma protein (“Rb”). p16 acts on Rb to inhibit the S phase, and Rb downregulates p16, creating negative feedback. Defective Rb fails to both inhibit the S phase and downregulate p16, thus resulting in overexpression of p16 in hyperproliferating cells (Romagosa, C. et al., p16^{Ink4a} overexpression in cancer: a tumor suppressor gene associated with senescence and high-grade tumors, Oncogene, Vol. 30, 2087-2097 (2011)).

Senescent cells are associated with secretion of many factors involved in intercellular signaling, including pro-inflammatory factors; secretion of these factors has been termed the senescence-associated secretory phenotype, or SASP (Freund, A. “Inflammatory networks during cellular senescence: causes and consequences” Trends Mol Med. 2010 May; 16(5):238-46). Autoimmune diseases, such as Crohn's disease and rheumatoid arthritis, are associated with chronic inflammation (Ferraccioli, G. et al. “Interleukin-1β and Interleukin-6 in Arthritis Animal Models: Roles in the Early Phase of Transition from Acute to Chronic Inflammation and Relevance for Human Rheumatoid Arthritis” Mol Med. 2010 November-December; 16(11-12): 552-557). Chronic inflammation may be characterized by the presence of pro-inflammatory factors at levels higher than baseline near the site of pathology, but lower than those found in acute inflammation. Examples of these factors include TNF, IL-1α, IL-1β, IL-5, IL-6, IL-8, IL-12, IL-23, CD2, CD3, CD20, CD22, CD52, CD80, CD86, C5 complement protein, BAFF, APRIL, IgE, α4β1 integrin and α4β7 integrin. Senescent cells also upregulate genes with roles in inflammation including IL-1β, IL-8, ICAM1, TNFAP3, ESM1 and CCL2 (Burton, D. G. A. et al., “Microarray analysis of senescent vascular smooth muscle cells: a link to atherosclerosis and vascular calcification”, Experimental Gerontology, Vol. 44, No. 10, pp. 659-665 (October 2009)).

Senescent cells secrete reactive oxygen species (“ROS”) as part of the SASP. ROS are believed to play an important role in maintaining senescence of cells. The secretion of ROS creates a bystander effect, where senescent cells induce senescence in neighboring cells: ROS create the very cellular damage known to activate p16 expression, leading to senescence (Nelson, G., A senescent cell bystander effect: senescence-induced senescence, Aging Cell, Vo. 11, 345-349 (2012)). The p16/Rb pathway leads to the induction of ROS, which in turn activates the protein kinase C delta creating a positive feedback loop that further enhance ROS, helping maintain the irreversible cell cycle arrest; it has even been suggested that exposing cancer cells to ROS might be effective to treat cancer by inducing cell phase arrest in hyperproliferating cells (Rayess, H. et al., Cellular senescence and tumor suppressor gene p16, Int J Cancer, Vol. 130, 1715-1725 (2012)).

Recent research demonstrates the therapeutic benefits of removing senescent cells. In vivo animal studies at the Mayo Clinic in Rochester, Minn., found that elimination of senescent cells in transgenic mice carrying a biomarker for elimination delayed age-related disorders associated with cellular senescence. Eliminating senescent cells in fat and muscle tissues substantially delayed the onset of sarcopenia and cataracts and reduced senescence indicators in skeletal muscle and the eye (Baker, D. J. et al., “Clearance of p16^{Ink4a}-positive senescent cells delays ageing-associated disorders”, Nature, Vol. 479, pp. 232-236, (2011)). Mice that were treated to induce senescent cell elimination were found to have larger diameters of muscle fibers as compared to untreated mice. Treadmill exercise tests indicated that treatment also preserved muscle function. Continuous treatment of transgenic mice for removal of senescent cells had no negative side effects and selectively delayed age-related phenotypes that depend on cells. This data demonstrates that removal of senescent cells produces beneficial therapeutic effects and shows that these benefits may be achieved without adverse effects.

Additional in vivo animal studies in mice found that removing senescent cells using senolytic agents treats aging-related disorders, atherosclerosis and pulmonary fibrosis. Short-term treatment with senolytic drugs in chronologically aged or progeroid mice alleviated several aging-related phenotypes (Zhu, Y. et al., “The Achilles' heel of senescent cells: from transcriptome to senolytic drugs”, Aging Cell, vol. 14, pp. 644-658 (2015)). Long-term treatment with senolytic drugs improved vasomotor function in mice with established atherosclerosis and reduced intimal plaque calcification (Roos, C. M. et al., “Chronic senolytic treatment alleviates established vasomotor dysfunction in aged or atherosclerotic mice”, Aging Cell (2016)). Removing senescent cells by administering senolytic agents reversed radiation-induced pulmonary fibrosis in mice (Pan, J. et al., “Inhibition of Bcl-2/xl with ABT-263 selectively kills senescent type II pneumocytes and reverses pulmonary fibrosis induced by ionizing radiation in mice”, International Journal of Radiation Oncology Biology Physics, Vol. 99, No. 2, pp. 353-361 (2017)). This data further demonstrates the benefits of removing senescent cells.

Senescent cells may also be specifically targeted and removed using antibodies that bind to cell-surface markers of senescence, such as AGE-modified proteins or AGE-modified peptides. Antibodies are Y-shaped proteins composed of two heavy chains and two light chains. The two arms of the Y shape form the fragment antigen-binding (Fab) region while the base or tail of the Y shape forms the fragment crystallizable (Fc) region of the antibody. Antigen binding occurs at the terminal portion of the fragment antigen-binding region (the tips of the arms of the Y shape) at a location referred to as the paratope, which is a set of complementarity determining regions (also known as CDRs or the hypervariable region). The complementarity determining regions vary among different antibodies and gives a given antibody its specificity for binding to a given antigen. The fragment crystallizable region of the antibody determines the result of antigen binding and may interact with the immune system, such as by triggering the complement cascade or initiating antibody-dependent cell-mediated cytotoxicity (ADCC). Antibody-based immunotherapies are particularly desirable because of their ability to specifically target and kill cells that express the antigen to which the antibody binds while sparing cells that do not express the antigen.

Antibodies that bind to advanced glycation end-products (anti-AGE antibodies) have been shown to be effective at treating various AGE disorders including sarcopenia (WO 2009/143411, US 2016/0215043, US 2016/0175413), atherosclerosis (US 2013/0243785), inflammation and auto-immune disorders (WO 2016/044252), neurodegenerative disorders (WO 2017/181116) and cancer (WO 2017/143073). Antibodies that bind to AGE-modified cells are known in the art. Examples include those described in U.S. Pat. No. 5,702,704 to Bucala and U.S. Pat. No. 6,380,165 to Al-Abed et al. Anti-AGE antibodies are commercially available, although the commercially available antibodies are not intended for therapeutic use.

Ultrasound is a mechanical technique that also is effective at specifically targeting cells. Ultrasound specifically targets cells based on physical differences in cell structure. Ultrasound may damage or destroy targeted cells or tissues by thermal effects (heating) or non-thermal effects such as cavitation, microstreaming and acoustic streaming. Non-destructive ultrasound also may provide therapeutic benefits such as interacting with the inflammatory process (Aviles Jr., F. et al., “Contact low-frequency ultrasound used to accelerate granulation tissue proliferation and rapid removal of nonviable tissue in colonized wounds: a case study”, Ostomy Wound Management, available online at www.o-wm.com/content/contact-low-frequency-ultrasound-used-accelerate-granulation-tissue-proliferation-and-rapid (2011)). Ultrasound parameters such as frequency, intensity, wavelength, velocity, wave shape, continuity (pulsed or constant application) and duration of application may be varied to provide a specific therapeutic effect.

Ultrasound is particularly effective in targeting cancerous cells and tumors. Malignant cells are more susceptible to damage by ultrasound than normal healthy cells (Lejbkowicz, F. et al., “Distinct sensitivity of normal and malignant cells to ultrasound in vitro”, Environmental Health Perspectives, Vol. 105, Supplement 6, pp. 1575-1578 (1997)). Low-intensity ultrasound may be used therapeutically to treat cancer in forms such as sonodynamic therapy (ultrasound administered with sensitizing molecules), ultrasound-mediated chemotherapy, sonoporation, ultrasound-mediated gene delivery and anti-vascular ultrasound therapy (Wood, A. K. W. et al., “A review of low-intensity ultrasound for cancer therapy”, Ultrasound in Medicine & Biology, Vol. 41, No. 4, pp. 905-928 (2015)).

Ultrasound has been shown to be effective against various types of cancer. Applying continuous ultrasound at a frequency of 1.1 MHz causes intensity-dependent cell membrane damage and cell viability loss in human leukemia cells (Wang, P. et al., “Membrane damage effect of continuous wave ultrasound on K562 human leukemia cells”, Journal of Ultrasound Medicine, Vol. 31, pp. 1977-1986 (2012)). Low frequency and intensity ultrasound can induce apoptosis in glioma cells both in vitro and in vivo (Zhang, Z. et al., “Low frequency and intensity ultrasound induces apoptosis of brain glioma in rats mediated by caspase-3 Bcl-2, and survivin”, Brain Research, Vol. 1473, pp. 25-34 (2012)). Sonodynamic therapy using porphyrin derivatives including 5-aminolevulinic acid, protoporphyrin IX and talaporfin sodium as sonosensitizers is cytotoxic to malignant glioma cells in vitro (Endo, S. et al., “Porphyrin derivatives-mediated sonodynamic therapy for malignant gliomas in vitro”, Ultrasound in Medicine and Biology, Vol. 41, No. 9, pp. 2458-2465 (2015)).

In addition to damaging cells and tissues directly, ultrasound may be administered in combination with other therapeutic agents. Ultrasound-mediated cavitation does not decrease the activity of small molecule, antibody or viral-based medicines (Myers, R. et al., “Ultrasound-mediated cavitation does not decrease the activity of small molecule, antibody or viral-based medicines”, International Journal of Nanomedicine, Vol. 13, pp. 337-349 (2018)). Ultrasound may be used to facilitate or enhance the delivery of therapeutic agents into target cells or tissues. Ultrasound increases accumulation of liposomal nanoparticles and tumor permeability in epithelial tumors (Watson, K. D. et al., “Ultrasound increases nanoparticle delivery by reducing intratumoral pressure and increasing transport in epithelial and epithelial-mesenchymal transition tumors”, Cancer Research, Vol. 72, No. 6, pp. 1485-1493 (2012)). Similarly, sonodynamic therapy increases the uptake of titanium dioxide nanoparticles modified with a protein specific for cancer cells into the targeted cells (Ninomiya, K. et al., “Targeted sonodynamic therapy using protein-modified TiO₂ nanoparticles”, Ultrasonics Sonochemistry, Vol. 19, pp. 607-614 (2012)).

Ultrasound also enhances the delivery and activity of larger therapeutic agents such as antibodies. Ultrasound can dramatically accelerate antigen binding to immobilized antibodies (Chen, R. et al., “Ultrasound-accelerated immunoassay, as exemplified by enzyme immunoassay of choriogonadotropin”, Clinical Chemistry, Vol. 30, No. 9, pp. 1446-1451 (1984)). Pulsed high-intensity focused ultrasound enhances the delivery of a murine IgG1_{K} monoclonal antibody in human epidermoid tumors (Khaibullina, A. et al., “Pulsed high-intensity focused ultrasound enhances uptake of radiolabeled monoclonal antibody to human epidermoid tumor in nude mice”, Journal of Nuclear Medicine, Vol. 49, pp. 295-302 (2008)). Pulsed ultrasound administered in combination with microbubbles enhances the delivery of anti-epidermal growth factor receptor (EGFR) antibodies to glioma tumor cells in a mouse model (Liao, A-H. et al., “Enhanced therapeutic epidermal growth factor receptor (EGFR) antibody delivery via pulsed ultrasound with targeting microbubbles for glioma treatment”, Journal of Medical and Biological Engineering, Vol. 35, pp. 156-164 (2015)). Low-intensity ultrasound enhances the anticancer activity of cetuximab (an anti-EGFR antibody) on killing human head and neck cancer cells in vitro (Masui, T. et al., “Low-intensity ultrasound enhances the anticancer activity of cetuximab in human head and neck cancer cells”, Experimental and Therapeutic Medicine, Vol. 5, pp. 11-16 (2013)).

A significant limitation of pharmacotherapy is drug delivery into certain regions of the body known as “restricted sites”. It is challenging or impossible to deliver therapeutic agents to restricted sites such as the brain, joints, prostate, testes and eyes. Access may be restricted due to a blood-tissue barrier, such as the blood-brain barrier, blood-testis barrier, blood-ocular barrier, blood-retinal barrier or blood-aqueous barrier. For example, the blood-brain barrier separates the brain and central nervous system from circulating blood, which restricts the ability of therapeutic agents to access the brain. Access may also be restricted by tissue such as the synovial membrane, which restricts access to joints. The synovial membrane filters most therapeutic agents in the intrasynovial joint space (Allen, K. D. et al., “Evaluating intra-articular drug delivery for the treatment of osteoarthritis in a rat model”, Tissue Engineering: Part B, Vol. 16, No. 1, pp. 81-92 (2010)). It is particularly difficult to deliver large therapeutic agents, such as antibodies, to restricted sites.

### SUMMARY

In a first aspect, the invention is a method of killing AGE-modified cells comprising applying ultrasound to a subject and administering to the subject a composition comprising an anti-AGE antibody.

In a second aspect, the invention is a method of killing AGE-modified cells in a tissue culture or cell culture comprising applying ultrasound to the tissue culture or cell culture and administering to the tissue culture or cell culture a composition comprising an anti-AGE antibody.

In a third aspect, the invention is a method of treating an AGE disorder comprising applying ultrasound to a subject having an AGE disorder and administering to the subject a composition comprising an anti-AGE antibody.

In a fourth aspect, the invention is a method of treating a subject with a glioma comprising disrupting the blood-brain barrier of the subject, followed by administering to the subject a composition comprising an anti-AGE antibody.

In a fifth aspect, the invention is a method of treating a subject with osteoarthritis comprising applying ultrasound to the affected joint, followed by administering to the subject a composition comprising an anti-AGE antibody. The composition is administered intravenously.

In a sixth aspect, the invention is a method of treating an AGE disorder comprising administering to a subject an anti-AGE antibody conjugated to a microbubble and applying ultrasound to the subject.

In a seventh aspect, the invention is a composition comprising an anti-AGE antibody conjugated to a microbubble.

In an eighth aspect, the invention is a composition comprising an anti-AGE antibody conjugated to a microbubble for use in treating an AGE disorder.

In a ninth aspect, the invention is use of an anti-AGE antibody conjugated to a microbubble for the manufacture of a medicament for treating an AGE disorder.

### Definitions

The term “restricted site” means a location of the human body that is difficult for therapeutic agents to access. Examples of restricted sites include the brain, joints, eyes, testes and prostate.

The term “peptide” means a molecule composed of 2-50 amino acids.

The term “protein” means a molecule composed of more than 50 amino acids.

The terms “advanced glycation end-product”, “AGE”, “AGE-modified protein or peptide” and “glycation end-product” refer to modified proteins or peptides that are formed as the result of the reaction of sugars with protein side chains that further rearrange and form irreversible cross-links. This process begins with a reversible reaction between a reducing sugar and an amino group to form a Schiff base, which proceeds to form a covalently-bonded Amadori rearrangement product. Once formed, the Amadori product undergoes further rearrangement to produce AGEs. AGE-modified proteins and antibodies to AGE-modified proteins are described in U.S. Pat. No. 5,702,704 to Bucala (“Bucala”) and U.S. Pat. No. 6,380,165 to Al-Abed et al. (“Al-Abed”). Glycated proteins or peptides that have not undergone the necessary rearrangement to form AGEs, such as N-deoxyfructosyllysine found on glycated albumin, are not AGEs. AGEs may be identified by the presence of AGE modifications (also referred to as AGE epitopes or AGE moieties) such as 2-(2-furoyl)-4(5)-(2-furanyl)-1H-imidazole (“FFI”); 5-hydroxymethyl-1-alkylpyrrole-2-carbaldehyde (“Pyrraline”); 1-alkyl-2-formyl-3,4-diglycosyl pyrrole (“AFGP”), a non-fluorescent model AGE; carboxymethyllysine; carboxyethyllysine; and pentosidine. ALI, another AGE, is described in Al-Abed.

The term “AGE antigen” means a substance that elicits an immune response against an AGE-modified protein or peptide of a cell. The immune response against an AGE-modified protein or peptide of a cell does not include the production of antibodies to the non-AGE-modified protein or peptide.

“An antibody that binds to an AGE-modified protein on a cell”, “antibody that binds to an AGE-modified cell”, “anti-AGE antibody” or “AGE antibody” means an antibody, antibody fragment or other protein or peptide that binds to an AGE-modified protein or peptide which preferably includes a constant region of an antibody, where the protein or peptide which has been AGE-modified is a protein or peptide normally found bound on the surface of a cell, preferably a mammalian cell, more preferably a human, cat, dog, horse, camelid (for example, camel or alpaca), cattle, sheep, pig, or goat cell. “An antibody that binds to an AGE-modified protein on a cell”, “antibody that binds to an AGE-modified cell”, “anti-AGE antibody” or “AGE antibody” does not include an antibody or other protein which binds with the same specificity and selectivity to both the AGE-modified protein or peptide, and the same non-AGE-modified protein or peptide (that is, the presence of the AGE modification does not increase binding). AGE-modified albumin is not an AGE-modified protein on a cell, because albumin is not a protein normally found bound on the surface of cells. “An antibody that binds to an AGE-modified protein on a cell”, “antibody that binds to an AGE-modified cell”, “anti-AGE antibody” or “AGE antibody” only includes those antibodies which lead to removal, destruction, or death of the cell. Also included are antibodies which are conjugated, for example to a toxin, drug, or other chemical or particle. Preferably, the antibodies are monoclonal antibodies, but polyclonal antibodies are also possible.

The term “senescent cell” means a cell which is in a state of proliferative arrest and expresses one or more biomarkers of senescence, such as activation of p16^{Ink4a} or expression of senescence-associated β-galactosidase. Also included are cells which express one or more biomarkers of senescence, do not proliferate in vivo, but may proliferate in vitro under certain conditions, such as some satellite cells found in the muscles of ALS patients.

The term “senolytic agent” means a small molecule with a molecular weight of less than 900 daltons that destroys senescent cells. The term “senolytic agent” does not include antibodies, antibody conjugates, proteins, peptides or biologic therapies.

The term “AGE disorder” means a pathological condition, disease or disorder associated with advanced glycation end-products (AGEs), AGE-modified cells or cellular senescence.

The term “variant” means a nucleotide, protein or amino acid sequence different from the specifically identified sequences, wherein one or more nucleotides, proteins or amino acid residues is deleted, substituted or added. Variants may be naturally-occurring allelic variants, or non-naturally-occurring variants. Variants of the identified sequences may retain some or all of the functional characteristics of the identified sequences.

The term “percent (%) sequence identity” is defined as the percentage of amino acid residues in a candidate sequence that are identical to the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using publicly available computer software such as BLAST, BLAST-2, ALIGN or Megalign (DNASTAR) software. Preferably, % sequence identity values are generated using the sequence comparison computer program ALIGN-2. The ALIGN-2 sequence comparison computer program is publicly available from Genentech, Inc. (South San Francisco, Calif.), or may be compiled from the source code, which has been filed with user documentation in the U.S. Copyright Office and is registered under U.S. Copyright Registration No. TXU510087. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. Where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained using the ALIGN-2 computer program.

### BRIEF DESCRIPTION OF THE DRAWING

FIG. 1 is a graph of the response versus time in an antibody binding experiment.

FIG. 2A illustrates the binding of an anti-AGE antibody to pancreatic cancer cells from the human pancreatic cancer PANG-1 cell line.

FIG. 2B illustrates the binding of an anti-AGE antibody to pancreatic cancer cells from the human pancreatic cancer PANC-1 cell line.

FIG. 2C illustrates the binding of an anti-AGE antibody to pancreatic cancer cells from the human pancreatic cancer PANG-1 cell line.

FIG. 2D illustrates the binding of an anti-AGE antibody to pancreatic cancer cells from the human pancreatic cancer PANG-1 cell line.

FIG. 3 illustrates the binding of an anti-AGE antibody to glioma cells from the HTB-14 glioblastoma cell line.

FIG. 4A illustrates the binding of an anti-AGE antibody to glioma cells from the HTB-15 glioblastoma cell line.

FIG. 4B illustrates the binding of an anti-AGE antibody to glioma cells from the HTB-15 glioblastoma cell line.

FIG. 4C illustrates the binding of an anti-AGE antibody to glioma cells from the HTB-15 glioblastoma cell line.

### DETAILED DESCRIPTION

It has been discovered that ultrasound can allow temporary access to restricted sites. Applying diagnostic ultrasound in combination with administration of microbubbles disrupts the blood-brain barrier (Zhao, B. et al., “Blood-brain barrier disruption induced by diagnostic ultrasound combined with microbubbles in mice”, Oncotarget, Vol. 9, No. 4, pp. 4897-4914 (2018)). Focused ultrasound applied in the presence of microbubbles disrupts the blood-brain barrier to allow delivery of the chemotherapy drugs 1,3-bis(2-chloroethyl)-1-nitrosourea (BCNU or carmustine) and doxorubicin to glioblastomas in rats (Liu, H-L. et al., “Blood-brain barrier disruption with focused ultrasound enhances delivery of chemotherapeutic drugs for glioblastoma treatment”, Radiology, Vol. 255, No. 2, pp. 415-425 (2010); Sun, T. et al., “Closed-loop control of targeted ultrasound drug delivery across the blood-brain/tumor barriers in a rat glioma model”, Proceedings of the National Academy of Sciences, pp. 1-10 (2017)). Ultrasound applied in the presence of microbubbles is able to disrupt the blood-brain barrier to allow single chain antibodies and the anti-EGFR monoclonal antibody trastuzumab (HERCEPTIN®) to access the brains of mice (Nisbet, R. M. et al., “Combined effects of scanning ultrasound and a tau-specific single chain antibody in a tau transgenic mouse model”, Brain, Vol. 140, pp. 1220-1230 (2017); Kinoshita, M. et al., “Noninvasive localized delivery of Herceptin to the mouse brain by MRI-guided focused ultrasound-induced blood-brain barrier disruption”, Proceedings of the National Academy of Sciences, Vol. 103, No. 31, pp. 11719-11723 (2006)). These studies suggest that a combination therapy including application of ultrasound and administration of antibodies is an effective treatment regimen for targeting cells and tissues in restricted sites.

A number of AGE disorders affect restricted sites. For example, recent studies have identified an association between cellular senescence and gliomas. Glioma cells become spontaneously senescent in vitro (Stoczynska-Fidelus, E., et al., “Spontaneous in vitro senescence of glioma cells confirmed by an antibody against IDH1^{R132H}”, Anticancer Research, Vol. 34, pp. 2859-2868 (2014)). Similarly, advanced glycation end-products cause a dose-dependent increase of nitrite accumulation in glioma cells in vitro, which results in increased nitric oxide release and a corresponding increase in oxidative damage (Lin, C-H. et al., “Advanced glycosylation end products induce nitric oxide synthase expression in C6 glioma cells involvement of a p38 MAP kinase-dependent mechanism”, Life Sciences, Vol. 69, pp. 2503-2515 (2001)). In addition, glioma invasiveness is accompanied by reduced cellular proliferative activity, a sign of senescence (Berens, M. E. et al., “‘. . . those left behind.’ Biology and oncology of invasive glioma cells”, Neoplasia, Vol. 1, No. 3, pp. 208-219 (1999)). By identification of a common link between cellular senescence and gliomas, the Present Application shows that antibodies that bind to advanced glycation end-products are an effective treatment for gliomas. Application of ultrasound may be used to disrupt the blood-brain barrier and permit anti-AGE antibodies to target and kill glioma cells.

The present invention uses a combination of ultrasound and anti-AGE antibodies to specifically target and kill cells expressing AGE-modified proteins or peptides (AGE-modified cells) in a restricted site. The ultrasound provides access to the restricted site, which then allows the anti-AGE antibodies to access AGE-modified cells within the restricted site. The ultrasound provides access to the restricted site with or without contrast media such as microbubbles.

The combination of ultrasound and anti-AGE antibodies may also be used to enhance the destruction of AGE-modified cells. Ultrasound increases antibody binding to a target antigen, which will improve the binding of anti-AGE antibodies to AGE-modified cells. Once bound, ultrasound also enhances cell killing through the antibody-dependent cell-mediated cytotoxicity (ADCC) mechanism. In addition, AGE-modified cells that have bound to anti-AGE antibodies are more susceptible to destruction by ultrasound. The ability of ultrasound to interfere with inflammatory processes will also reduce inflammation, a key driver of cellular senescence. The combination of ultrasound and anti-AGE antibodies is synergistic and produces a greater than additive effect at killing AGE-modified cells, such as senescent cells. These enhanced cell-killing effects may be realized in any location of the body or in in vitro settings.

A method of killing AGE-modified cells includes applying ultrasound and administering an antibody that binds to a glycation end-product (anti-AGE antibody). The ultrasound may be applied before the antibodies are administered to facilitate access to a restricted site. Alternatively, the ultrasound may be applied after the antibodies are administered and/or during administration.

The AGE-modified cells may be found in any part of the body. Preferably, the AGE-modified cells are in a restricted site. Examples of restricted sites include the brain, joints, eyes, testes and prostate. Alternatively, the AGE-modified cells may be in an in vitro setting, such as a tissue culture or cell culture.

The ultrasound may be applied at a frequency of 20,000 Hz-15 MHz. Preferably, the ultrasound is applied at a frequency of 0.5-10 MHz, including 0.6 MHz, 0.7 MHz, 0.8 MHz, 0.9 MHz, 1.0 MHz, 1.5 MHz, 2.0 MHz, 2.5 MHz, 3.0 MHz, 3.5 MHz, 4.0 MHz, 4.5 MHz, 5.0 MHz, 5.1 MHz, 5.2 MHz, 5.3 MHz, 5.4 MHz, 5.5 MHz, 5.6 MHz, 5.7 MHz, 5.8 MHz, 5.9 MHz, 6.0 MHz, 6.1 MHz, 6.2 MHz, 6.3 MHz, 6.4 MHz, 6.5 MHz, 6.6 MHz, 6.7 MHz, 6.8 MHz, 6.9 MHz, 7.0 MHz, 7.5 MHz, 8.0 MHz, 8.5 MHz, 9.0 MHz and 9.5 MHz.

The ultrasound may be applied at an intensity of 0.01-5 W/cm². Preferably, the ultrasound is applied at an intensity of 0.05-5 W/cm², including 0.06 W/cm², 0.07 W/cm², 0.08 W/cm², 0.09 W/cm², 0.1 W/cm², 0.2 W/cm², 0.3 W/cm², 0.4 W/cm², 0.5 W/cm², 0.6 W/cm², 0.7 W/cm², 0.8 W/cm², 0.9 W/cm², 1.0 W/cm², 1.1 W/cm², 1.2 W/cm², 1.3 W/cm², 1.4 W/cm², 1.5 W/cm², 1.6 W/cm², 1.7 W/cm², 1.8 W/cm², 1.9 W/cm², 2.0 W/cm², 2.5 W/cm², 3.0 W/cm², 3.5 W/cm², 4.0 W/cm² and 4.5 W/cm².

The ultrasound may be continuous or may be pulsed. Preferably, the ultrasound is pulsed ultrasound. Pulsed ultrasound may have a pulse repetition frequency (PRF) of 0.01-20 kHz. Preferably, the pulsed ultrasound has a PRF of 0.05-10 kHz, including 0.06 kHz, 0.07 kHz, 0.08 kHz, 0.09 kHz, 0.1 kHz, 0.2 kHz, 0.3 kHz, 0.4 kHz, 0.5 kHz, 1.0 kHz, 1.5 kHz, 2.0 kHz, 3.0 kHz, 3.5 kHz, 4.0 kHz, 4.5 kHz, 5.0 kHz, 5.5 kHz, 6.0 kHz, 6.5 kHz, 7.0 kHz, 7.5 kHz, 8.0 kHz, 8.5 kHz, 9.0 kHz and 9.5 kHz.

The ultrasound may be applied once or multiple times per day. For example, the ultrasound may be applied 1-100 times per day. The ultrasound may be applied at regular intervals, such as once per hour (24 times per day), or may be applied multiple times within a fixed time period, such as 60 times per hour. Less frequent administration is also possible, including once per week or once per month.

The ultrasound may be focused or unfocused. Focused ultrasound typically delivers higher intensities than unfocused ultrasound.

Other ultrasound parameters, such as wavelength, velocity, wave shape, duration of application, pulse duration, spatial pulse length and duty factor, may be varied to provide a specific therapeutic effect. Preferably, the ultrasound parameters are selected so as to target AGE-modified cells while sparing other cells.

Appropriate ultrasound parameters may be determined by simple experimentation. Cells may be isolated, such as in a sample from a patient or in a cell culture. Physical differences between cells, such as stiffening or changes in elasticity, allows target cells to be identified (see, for example, U.S. Pat. Nos. 6,067,859 and 7,751,057). Ultrasound may be applied to the cells until a desired outcome is achieved. The cells are observed before and after ultrasound application to determine the effect of the ultrasound. One or more ultrasound parameters may be varied and the ultrasound reapplied. The cells are then observed to determine the effect of the modified ultrasound parameters. This process may be repeated until a desired therapeutic effect is achieved. For example, AGE-modified cells may be isolated and ultrasound may be applied to determine the resonant harmonic frequency that will result in cell destruction. The frequency of the ultrasound may be gradually increased until the resonant harmonic frequency is determined. Determining appropriate ultrasound parameters is a simple process for a skilled artisan since ultrasound is a well-studied mechanical technique that produces predictable results.

Contrast media may optionally be administered prior to or simultaneously with application of the ultrasound. Preferred contrast media include gas-filled microbubbles. Microbubbles may have a shell composed of albumin (ALBUNEX®, BISPHERE®, ECHOGEN® and OPTISON®), phospholipids (DEFINITY®, IMAGENT®, MICROMARKER®, SONAZOID®, SONOVUE® and TARGESTAR®), galactolipids (LEVOVIST®) or PLGA/phospholipids (IMAGIFY®). Microbubbles that have a nanometer-scale diameter are also referred to as nanobubbles. Contrast media may be administered near the location where the ultrasound will be applied.

Microbubbles may also be used for ultrasonic drug delivery. Therapeutic agents may be conjugated to the exterior of the microbubbles or may be contained in the gas-filled interior of the microbubbles. Microbubbles may be conjugated to targeting ligands. Preferred targeting ligands include monoclonal antibodies, which may also act as therapeutic agents. The monoclonal antibodies are preferably humanized (or similarly modified as appropriate for the species being treated) to reduce their immunogenicity. A particularly preferred targeting ligand is a humanized monoclonal anti-AGE antibody. Other drug carriers for ultrasonic drug delivery include liposomes and micelles. Liposomes and micelles may also be conjugated to monoclonal antibodies, such as anti-AGE antibodies.

The ultrasound may be generated and applied using any suitable ultrasound transducer. A preferred ultrasound transducer is an ultrasound probe. A water-based gel may be applied to the area of treatment to improve transmission of ultrasound into the body. Alternatively, ultrasound generators may be implanted in the body to further facilitate access to a restricted site (see, for example, U.S. Pat. No. 8,977,361).

The anti-AGE antibody may bind to one or more AGE-modified proteins or peptides having an AGE modification such as FFI, pyrraline, AFGP, ALI, carboxymethyllysine (CML), carboxyethyllysine (CEL) and pentosidine, and mixtures of such antibodies. Preferably, the antibody is non-immunogenic to the animal in which it will be used, such as non-immunogenic to humans; companion animals including cats, dogs and horses; and commercially important animals, such camels (or alpaca), cattle (bovine), sheep, pig, and goats. More preferably, the antibody has the same species constant region as antibodies of the animal to reduce the immune response against the antibody, such as being humanized (for humans), felinized (for cats), caninized (for dogs), equuinized (for horses), camelized (for camels or alpaca), bovinized (for cattle), ovinized (for sheep), porcinized (for pigs), or caperized (for goats). Most preferably, the antibody is identical to that of the animal in which it will be used (except for the variable region), such as a human antibody, a cat antibody, a dog antibody, a horse antibody, a camel antibody, a bovine antibody, a sheep antibody, a pig antibody, or a goat antibody. Details of the constant regions and other parts of antibodies for these animals are described below. The antibody may be monoclonal or polyclonal. Preferably, the antibody is a monoclonal antibody.

Preferred anti-AGE antibodies include those which bind to proteins or peptides that exhibit a carboxymethyllysine or carboxyethyllysine AGE modification. Carboxymethyllysine (also known as N(epsilon)-(carboxymethyl)lysine, N(6)-carboxymethyllysine, or 2-Amino-6-(carboxymethylamino)hexanoic acid) and carboxyethyllysine (also known as N-epsilon-(carboxyethyl)lysine) are found on proteins or peptides and lipids as a result of oxidative stress and chemical glycation. CML- and CEL-modified proteins or peptides are recognized by the receptor RAGE which is expressed on a variety of cells. CML and CEL have been well-studied and CML- and CEL-related products are commercially available. For example, Cell Biolabs, Inc. sells CML-BSA antigens, CML polyclonal antibodies, CML immunoblot kits, and CML competitive ELISA kits (www.cellbiolabs.com/cml-assays) as well as CEL-BSA antigens and CEL competitive ELISA kits (www.cellbiolabs.com/cel-n-epsilon-carboxyethyl-lysine-assays-and-reagents). A preferred antibody includes the variable region of the commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, Minn.; catalog no. MAB3247), modified to have a human constant region (or the constant region of the animal into which it will be administered). Commercially-available antibodies, such as the carboxymethyl lysine antibody corresponding to catalog no. MAB3247 from R&D Systems, Inc., may be intended for diagnostic purposes and may contain material that is not suited for use in animals or humans. Preferably, commercially-available antibodies are purified and/or isolated prior to use in animals or humans to remove toxins or other potentially-harmful material.

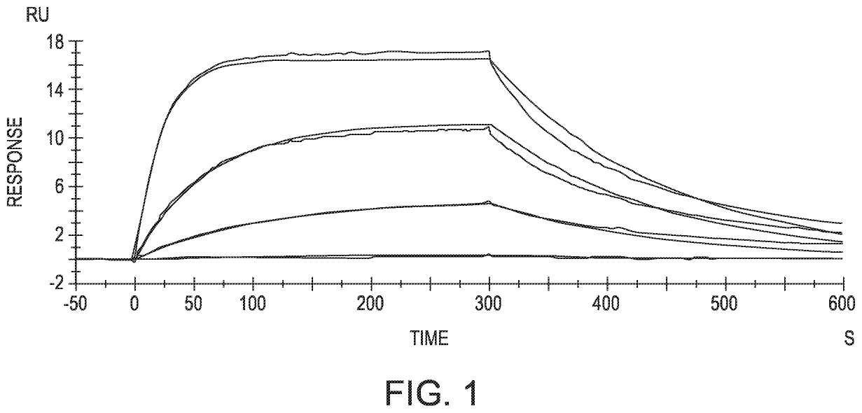

The anti-AGE antibody preferably has a low rate of dissociation from the antibody-antigen complex, or k_{d} (also referred to as k_{back} or off-rate), preferably at most 9×10⁻⁶, 8×10⁻³, 7×10⁻³ or 6×10 (sec⁻¹). The anti-AGE antibody preferably has a high affinity for the AGE-modified protein of a cell, which may be expressed as a low dissociation constant K_{D} of at most 9×10⁻⁶, 8×10⁻⁶, 7×10⁻⁶, 6×10⁻⁶, 5×10⁻⁶, 4×10⁻⁶ or 3×10⁻⁶ (M). Preferably, the binding properties of the anti-AGE antibody are similar to, the same as, or superior to the carboxymethyl lysine MAb (Clone 318003) available from R&D Systems, Inc. (Minneapolis, Minn.; catalog no. MAB3247), illustrated in FIG. 1.

The anti-AGE antibody may destroy AGE-modified cells through antibody-dependent cell-mediated cytotoxicity (ADCC). ADCC is a mechanism of cell-mediated immune defense in which an effector cell of the immune system actively lyses a target cell whose membrane-surface antigens have been bound by specific antibodies. ADCC may be mediated by natural killer (NK) cells, macrophages, neutrophils or eosinophils. The effector cells bind to the Fc portion of the bound antibody. The anti-AGE antibody may also destroy AGE-modified cells through complement-dependent cytotoxicity (CDC). In CDC, the complement cascade of the immune system is triggered by an antibody binding to a target antigen.

The anti-AGE antibody may be conjugated to an agent that causes the destruction of AGE-modified cells. Such agents may be a toxin, a cytotoxic agent, magnetic nanoparticles, and magnetic spin-vortex discs.

A toxin, such as pore-forming toxins (PFT) (Aroian R. et al., “Pore-Forming Toxins and Cellular Non-Immune Defenses (CNIDs),” Current Opinion in Microbiology, 10:57-61 (2007)), conjugated to an anti-AGE antibody may be injected into a patient to selectively target and remove AGE-modified cells. The anti-AGE antibody recognizes and binds to AGE-modified cells. Then, the toxin causes pore formation at the cell surface and subsequent cell removal through osmotic lysis.

Magnetic nanoparticles conjugated to the anti-AGE antibody may be injected into a patient to target and remove AGE-modified cells. The magnetic nanoparticles can be heated by applying a magnetic field in order to selectively remove the AGE-modified cells.

As an alternative, magnetic spin-vortex discs, which are magnetized only when a magnetic field is applied to avoid self-aggregation that can block blood vessels, begin to spin when a magnetic field is applied, causing membrane disruption of target cells. Magnetic spin-vortex discs, conjugated to anti-AGE antibodies specifically target AGE-modified cell types, without removing other cells.

A humanized anti-AGE antibody according to the present invention may have the human constant region sequence of amino acids shown in SEQ ID NO: 22. The heavy chain complementarity determining regions of the humanized anti-AGE antibody may have one or more of the protein sequences shown in SEQ ID NO: 23 (CDR1H), SEQ ID NO: 24 (CDR2H) and SEQ ID NO: 25 (CDR3H). The light chain complementarity determining regions of the humanized anti-AGE antibody may have one or more of the protein sequences shown in SEQ ID NO: 26 (CDR1L), SEQ ID NO: 27 (CDR2L) and SEQ ID NO: 28 (CDR3L).

The heavy chain of a humanized anti-AGE antibody may have or may include the protein sequence of SEQ ID NO: 1. The variable domain of the heavy chain may have or may include the protein sequence of SEQ ID NO: 2. The complementarity determining regions of the variable domain of the heavy chain (SEQ ID NO: 2) are shown in SEQ ID NO: 41, SEQ ID NO: 42 and SEQ ID NO: 43. The kappa light chain of a humanized anti-AGE antibody may have or may include the protein sequence of SEQ ID NO: 3. The variable domain of the kappa light chain may have or may include the protein sequence of SEQ ID NO: 4. Optionally, the arginine (Arg or R) residue at position 128 of SEQ ID NO: 4 may be omitted. The complementarity determining regions of the variable domain of the light chain (SEQ ID NO: 4) are shown in SEQ ID NO: 44, SEQ ID NO: 45 and SEQ ID NO: 46. The variable regions may be codon-optimized, synthesized and cloned into expression vectors containing human immunoglobulin G1 constant regions. In addition, the variable regions may be used in the preparation of non-human anti-AGE antibodies.

The antibody heavy chain may be encoded by the DNA sequence of SEQ ID NO: 12, a murine anti-AGE immunoglobulin G2b heavy chain. The protein sequence of the murine anti-AGE immunoglobulin G2b heavy chain encoded by SEQ ID NO: 12 is shown in SEQ ID NO: 16. The variable region of the murine antibody is shown in SEQ ID NO: 20, which corresponds to positions 25-142 of SEQ ID NO: 16. The antibody heavy chain may alternatively be encoded by the DNA sequence of SEQ ID NO: 13, a chimeric anti-AGE human immunoglobulin G1 heavy chain. The protein sequence of the chimeric anti-AGE human immunoglobulin G1 heavy chain encoded by SEQ ID NO: 13 is shown in SEQ ID NO: 17. The chimeric anti-AGE human immunoglobulin includes the murine variable region of SEQ ID NO: 20 in positions 25-142. The antibody light chain may be encoded by the DNA sequence of SEQ ID NO: 14, a murine anti-AGE kappa light chain. The protein sequence of the murine anti-AGE kappa light chain encoded by SEQ ID NO: 14 is shown in SEQ ID NO: 18. The variable region of the murine antibody is shown in SEQ ID NO: 21, which corresponds to positions 21-132 of SEQ ID NO: 18. The antibody light chain may alternatively be encoded by the DNA sequence of SEQ ID NO: 15, a chimeric anti-AGE human kappa light chain. The protein sequence of the chimeric anti-AGE human kappa light chain encoded by SEQ ID NO: 15 is shown in SEQ ID NO: 19. The chimeric anti-AGE human immunoglobulin includes the murine variable region of SEQ ID NO: 21 in positions 21-132.

A humanized anti-AGE antibody according to the present invention may have or may include one or more humanized heavy chains or humanized light chains. A humanized heavy chain may be encoded by the DNA sequence of SEQ ID NO: 30, 32 or 34. The protein sequences of the humanized heavy chains encoded by SEQ ID NOs: 30, 32 and 34 are shown in SEQ ID NOs: 29, 31 and 33, respectively. A humanized light chain may be encoded by the DNA sequence of SEQ ID NO: 36, 38 or 40. The protein sequences of the humanized light chains encoded by SEQ ID NOs: 36, 38 and 40 are shown in SEQ ID NOs: 35, 37 and 39, respectively. Preferably, the humanized anti-AGE antibody maximizes the amount of human sequence while retaining the original antibody specificity. A complete humanized antibody may be constructed that contains a heavy chain having a protein sequence chosen from SEQ ID NOs: 29, 31 and 33 and a light chain having a protein sequence chosen from SEQ ID NOs: 35, 37 and 39.

Particularly preferred anti-AGE antibodies may be obtained by humanizing murine monoclonal anti-AGE antibodies. Murine monoclonal anti-AGE antibodies have the heavy chain protein sequence shown in SEQ ID NO: 47 (the protein sequence of the variable domain is shown in SEQ ID NO: 52) and the light chain protein sequence shown in SEQ ID NO: 57 (the protein sequence of the variable domain is shown in SEQ ID NO: 62). A preferred humanized heavy chain may have the protein sequence shown in SEQ ID NO: 48, SEQ ID NO: 49, SEQ ID NO: 50 or SEQ ID NO: 51 (the protein sequences of the variable domains of the humanized heavy chains are shown in SEQ ID NO: 53, SEQ ID NO: 54, SEQ ID NO: 55 and SEQ ID NO: 56, respectively). A preferred humanized light chain may have the protein sequence shown in SEQ ID NO: 58, SEQ ID NO: 59, SEQ ID NO: 60 or SEQ ID NO: 61 (the protein sequences of the variable domains of the humanized light chains are shown in SEQ ID NO: 63, SEQ ID NO: 64, SEQ ID NO: 65 and SEQ ID NO: 66, respectively). Preferably, a humanized anti-AGE monoclonal antibody is composed a heavy chain having a protein sequence selected from the group consisting of SEQ ID NO: 48, SEQ ID NO: 49, SEQ ID NO: 50 and SEQ ID NO: 51 and a light chain having a protein sequence selected from the group consisting of SEQ ID NO: 58, SEQ ID NO: 59, SEQ ID NO: 60 and SEQ ID NO: 61. Humanized monoclonal anti-AGE antibodies composed of these protein sequences may have better binding and/or improved activation of the immune system, resulting in greater efficacy.

The protein sequence of an antibody from a non-human species may be modified to include the variable domain of the heavy chain having the sequence shown in SEQ ID NO: 2 or the kappa light chain having the sequence shown in SEQ ID NO: 4. The non-human species may be a companion animal, such as the domestic cat or domestic dog, or livestock, such as cattle, the horse or the camel. Preferably, the non-human species is not the mouse. The heavy chain of the horse (Equus caballus) antibody immunoglobulin gamma 4 may have or may include the protein sequence of SEQ ID NO: 5 (EMBUGenBank accession number AY445518). The heavy chain of the horse (Equus caballus) antibody immunoglobulin delta may have or may include the protein sequence of SEQ ID NO: 6 (EMBUGenBank accession number AY631942). The heavy chain of the dog (Canis familiaris) antibody immunoglobulin A may have or may include the protein sequence of SEQ ID NO: 7 (GenBank accession number L36871). The heavy chain of the dog (Canis familiaris) antibody immunoglobulin E may have or may include the protein sequence of SEQ ID NO: 8 (GenBank accession number L36872). The heavy chain of the cat (Felis catus) antibody immunoglobulin G2 may have or may include the protein sequence of SEQ ID NO: 9 (DDBJ/EMBL/GenBank accession number KF811175).

Animals of the camelid family, such as camels (Camelus dromedarius and Camelus bactrianus), llamas (Lama glama, Lama pacos and Lama vicugna), alpacas (Vicugna pacos) and guanacos (Lama guanicoe), have a unique antibody that is not found in other mammals. In addition to conventional immunoglobulin G antibodies composed of heavy and light chain tetramers, camelids also have heavy chain immunoglobulin G antibodies that do not contain light chains and exist as heavy chain dimers. These antibodies are known as heavy chain antibodies, HCAbs, single-domain antibodies or sdAbs, and the variable domain of a camelid heavy chain antibody is known as the VHH. The camelid heavy chain antibodies lack the heavy chain CH1 domain and have a hinge region that is not found in other species. The variable region of the Arabian camel (Camelus dromedarius) single-domain antibody may have or may include the protein sequence of SEQ ID NO: 10 (GenBank accession number AJ245148). The variable region of the heavy chain of the Arabian camel (Camelus dromedarius) tetrameric immunoglobulin may have or may include the protein sequence of SEQ ID NO: 11 (GenBank accession number AJ245184).

In addition to camelids, heavy chain antibodies are also found in cartilaginous fishes, such as sharks, skates and rays. This type of antibody is known as an immunoglobulin new antigen receptor or IgNAR, and the variable domain of an IgNAR is known as the VNAR. The IgNAR exists as two identical heavy chain dimers composed of one variable domain and five constant domains each. Like camelids, there is no light chain.

The protein sequences of additional non-human species may be readily found in online databases, such as the International ImMunoGeneTics Information System (www.imgt.org), the European Bioinformatics Institute (www.ebi.ac.uk), the DNA Databank of Japan (ddbj.nig.ac.jp/arsa) or the National Center for Biotechnology Information (www.ncbi.nlm.nih.gov).

An anti-AGE antibody or a variant thereof may include a heavy chain having at least 90%, 91%, 92%, 93%, 94%, 95%, 96%, 97%, 98%, 99%, or 100% sequence identity to the amino acid sequence of SEQ ID NO: 1, SEQ ID NO: 16, SEQ ID NO: 17, SEQ ID NO: 29, SEQ ID NO: 31, SEQ ID NO: 33, SEQ ID NO: 47, SEQ ID NO: 48, SEQ ID NO: 49, SEQ ID NO: 50 or SEQ ID NO: 51, including post-translational modifications thereof. A heavy chain having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE.

An anti-AGE antibody or a variant thereof may include a heavy chain variable region having at least 90%, 91%, 92%, 93%, 94%, 95%, 96%, 97%, 98%, 99%, or 100% sequence identity to the amino acid sequence of SEQ ID NO: 2, SEQ ID NO: 20, SEQ ID NO: 23, SEQ ID NO: 24, SEQ ID NO: 25, SEQ ID NO: 41, SEQ ID NO: 42, SEQ ID NO: 43, SEQ ID NO: 52, SEQ ID NO: 53, SEQ ID NO: 54, SEQ ID NO: 55, or SEQ ID NO: 56, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

An anti-AGE antibody or a variant thereof may include a light chain having at least 90%, 91%, 92%, 93%, 94%, 95%, 96%, 97%, 98%, 99%, or 100% sequence identity to the amino acid sequence of SEQ ID NO: 3, SEQ ID NO: 18, SEQ ID NO: 19, SEQ ID NO: 35, SEQ ID NO: 37, SEQ ID NO: 39, SEQ ID NO: 57, SEQ ID NO: 58, SEQ ID NO: 59, SEQ ID NO: 60 or SEQ ID NO: 61, including post-translational modifications thereof. A light chain having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

An anti-AGE antibody or a variant thereof may include a light chain variable region having at least 90%, 91%, 92%, 93%, 94%, 95%, 96%, 97%, 98%, 99%, or 100% sequence identity to the amino acid sequence of SEQ ID NO: 4, SEQ ID NO: 21, SEQ ID NO: 26, SEQ ID NO: 27, SEQ ID NO: 28, SEQ ID NO: 44, SEQ ID NO: 45, SEQ ID NO: 46, SEQ ID NO: 62, SEQ ID NO: 63, SEQ ID NO: 64, SEQ ID NO: 65 or SEQ ID NO: 66, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

Alternatively, the antibody may have the complementarity determining regions of commercially available mouse anti-glycation end-product antibody raised against carboxymethyl lysine conjugated with keyhole limpet hemocyanin (CML-KLH), the carboxymethyl lysine MAb (Clone 318003) available from R&D Systems, Inc. (Minneapolis, Minn.; catalog no. MAB3247).

The antibody may have or may include constant regions which permit destruction of targeted cells by a subject's immune system.

Mixtures of antibodies that bind to more than one type AGE of AGE-modified proteins may also be used.

Bi-specific antibodies, which are anti-AGE antibodies directed to two different epitopes, may also be used. Such antibodies will have a variable region (or complementary determining region) from those of one anti-AGE antibody, and a variable region (or complementary determining region) from a different antibody.

Antibody fragments may be used in place of whole antibodies. For example, immunoglobulin G may be broken down into smaller fragments by digestion with enzymes. Papain digestion cleaves the N-terminal side of inter-heavy chain disulfide bridges to produce Fab fragments. Fab fragments include the light chain and one of the two N-terminal domains of the heavy chain (also known as the Fd fragment). Pepsin digestion cleaves the C-terminal side of the inter-heavy chain disulfide bridges to produce F(ab′)₂ fragments. F(ab′)₂ fragments include both light chains and the two N-terminal domains linked by disulfide bridges. Pepsin digestion may also form the Fv (fragment variable) and Fc (fragment crystallizable) fragments. The Fv fragment contains the two N-terminal variable domains. The Fc fragment contains the domains which interact with immunoglobulin receptors on cells and with the initial elements of the complement cascade. Pepsin may also cleave immunoglobulin G before the third constant domain of the heavy chain (C_{H}3) to produce a large fragment F(abc) and a small fragment pFc′. Antibody fragments may alternatively be produced recombinantly. Preferably, such antibody fragments are conjugated to an agent that causes the destruction of AGE-modified cells.

If additional antibodies are desired, they can be produced using well-known methods. For example, polyclonal antibodies (pAbs) can be raised in a mammalian host by one or more injections of an immunogen, and if desired, an adjuvant. Typically, the immunogen (and adjuvant) is injected in a mammal by a subcutaneous or intraperitoneal injection. The immunogen may be an AGE-modified protein of a cell, such as AGE-antithrombin III, AGE-calmodulin, AGE-insulin, AGE-ceruloplasmin, AGE-collagen, AGE-cathepsin B, AGE-albumin such as AGE-bovine serum albumin (AGE-BSA), AGE-human serum albumin and ovalbumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexominase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-low density lipoprotein, AGE-hexokinase, AGE-apo C-I, AGE-RNAse, AGE-hemoglobin such as AGE-human hemoglobin, AGE-low density lipoprotein (AGE-LDL) and AGE-collagen IV. AGE-modified cells, such as AGE-modified erythrocytes, whole, lysed, or partially digested, may also be used as AGE antigens. Examples of adjuvants include Freund's complete, monophosphoryl Lipid A synthetic-trehalose dicorynomycolate, aluminum hydroxide (alum), heat shock proteins HSP 70 or HSP96, squalene emulsion containing monophosphoryl lipid A, α2-macroglobulin and surface active substances, including oil emulsions, pleuronic polyols, polyanions and dinitrophenol. To improve the immune response, an immunogen may be conjugated to a polypeptide that is immunogenic in the host, such as keyhole limpet hemocyanin (KLH), serum albumin, bovine thyroglobulin, cholera toxin, labile enterotoxin, silica particles or soybean trypsin inhibitor. A preferred immunogen conjugate is AGE-KLH. Alternatively, pAbs may be made in chickens, producing IgY molecules.

Monoclonal antibodies (mAbs) may also be made by immunizing a host or lymphocytes from a host, harvesting the mAb-secreting (or potentially secreting) lymphocytes, fusing those lymphocytes to immortalized cells (for example, myeloma cells), and selecting those cells that secrete the desired mAb. Other techniques may be used, such as the EBV-hybridoma technique. Non-human antibodies may be made less immunogenic to humans by engineering the antibodies to contain a combination of non-human and human antibody components. A chimeric antibody may be produced by combining the variable region of a non-human antibody with a human constant region. A humanized antibody may be produced by replacing the complementarity determining regions (CDRs) of a human antibody with those of a non-human antibody. Similarly, antibodies may be made less immunogenic to other species by being substantially “ized” to a given animal, such as cat, dog, horse, camel or alpaca, cattle, sheep, pig, or goat, at the amino acid level. If desired, the mAbs may be purified from the culture medium or ascites fluid by conventional procedures, such as protein A-sepharose, hydroxyapatite chromatography, gel electrophoresis, dialysis, ammonium sulfate precipitation or affinity chromatography. Additionally, human monoclonal antibodies can be generated by immunization of transgenic mice containing a third copy IgG human trans-loci and silenced endogenous mouse Ig loci or using human-transgenic mice. Production of humanized monoclonal antibodies and fragments thereof can also be generated through phage display technologies.

A “pharmaceutically acceptable carrier” includes any and all solvents, dispersion media, coatings, antibacterial and antifungal agents, isotonic and absorption delaying agents, and the like, compatible with pharmaceutical administration. Preferred examples of such carriers or diluents include water, saline, Ringer's solutions and dextrose solution. Supplementary active compounds can also be incorporated into the compositions. Solutions and suspensions used for parenteral administration can include a sterile diluent, such as water for injection, saline solution, polyethylene glycols, glycerin, propylene glycol or other synthetic solvents; antibacterial agents such as benzyl alcohol or methyl parabens; antioxidants such as ascorbic acid or sodium bisulfate; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The antibodies may be administered by injection, such as by intravenous injection or locally, such as by intra-articular injection into a joint. Pharmaceutical compositions suitable for injection include sterile aqueous solutions or dispersions for the extemporaneous preparation of sterile injectable solutions or dispersion. Various excipients may be included in pharmaceutical compositions of antibodies suitable for injection. Suitable carriers include physiological saline, bacteriostatic water, CREMOPHOR EL® (BASF; Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. Various antibacterial and anti-fungal agents, for example, parabens, chlorobutanol, phenol, ascorbic acid, and thimerosal, can contain microorganism contamination. Isotonic agents such as sugars, polyalcohols, such as mannitol, sorbitol, and sodium chloride can be included in the composition. Compositions that can delay absorption include agents such as aluminum monostearate and gelatin. Sterile injectable solutions can be prepared by incorporating antibodies, and optionally other therapeutic components, in the required amount in an appropriate solvent with one or a combination of ingredients as required, followed by sterilization. Methods of preparation of sterile solids for the preparation of sterile injectable solutions include vacuum drying and freeze-drying to yield a solid.

For administration by inhalation, the antibodies may be delivered as an aerosol spray from a nebulizer or a pressurized container that contains a suitable propellant, for example, a gas such as carbon dioxide. Antibodies may also be delivered via inhalation as a dry powder, for example using the iSPERSE™ inhaled drug delivery platform (PULMATRIX, Lexington, Mass.). The use of anti-AGE antibodies which are chicken antibodies (IgY) may be non-immunogenic in a variety of animals, including humans, when administered by inhalation.

An appropriate dosage level of each type of antibody will generally be about 0.01 to 500 mg per kg patient body weight. Preferably, the dosage level will be about 0.1 to about 250 mg/kg; more preferably about 0.5 to about 100 mg/kg. A suitable dosage level may be about 0.01 to 250 mg/kg, about 0.05 to 100 mg/kg, or about 0.1 to 50 mg/kg. Within this range the dosage may be 0.05 to 0.5, 0.5 to 5 or 5 to 50 mg/kg. Although each type of antibody may be administered on a regimen of 1 to 4 times per day, such as once or twice per day, antibodies typically have a long half-life in vivo. Accordingly, each type of antibody may be administered once a day, once a week, once every two or three weeks, once a month, or once every 60 to 90 days.

Unit dosage forms can be created to facilitate administration and dosage uniformity. Unit dosage form refers to physically discrete units suited as single dosages for the subject to be treated, containing a therapeutically effective quantity of one or more types of antibodies in association with the required pharmaceutical carrier. Preferably, the unit dosage form is in a sealed container and is sterile.

A subject that receives application of ultrasound and administration of an anti-AGE antibody may be tested to determine if the treatment has been effective to kill AGE-modified cells. A subject may be considered to have received an effective treatment if he or she demonstrates a reduction in one or more symptoms of an AGE disorder between subsequent measurements or over time. For example, a subject with glioblastoma may be considered to have received an effective treatment based on a reduction in tumor size. Alternatively, the concentration and/or number of senescent cells may be measured over time. Treatment and subsequent testing may be repeated until the desired therapeutic result is achieved.

Any mammal may be treated by the methods herein described. Humans are a preferred mammal for treatment. Other mammals that may be treated include mice, rats, goats, sheep, pigs, cows, horses and companion animals, such as dogs or cats. Alternatively, any of the mammals or subjects identified above may be excluded from the patient population in need of treatment for pain associated with inflammation. The methods may also be applied in vitro, such as to a cell culture or tissue culture.

A subject may be identified as in need of treatment based on a diagnosis of having an AGE disorder. Examples of AGE disorders include Alzheimer's disease, amyotrophic lateral sclerosis (ALS or Lou Gehrig's Disease), chronic obstructive pulmonary disease (COPD), Huntington's chorea, idiopathic pulmonary fibrosis, muscular dystrophy (including Becker's, Duchenne, Limb-Girdle and Yamamoto's muscular dystrophy), macular degeneration, cataracts, diabetic retinopathy, Parkinson's disease, progeria (including Werner Syndrome and Hutchinson Gilford progeria), vitiligo, cystic fibrosis, atopic dermatitis, eczema, arthritis (including osteoarthritis, rheumatoid arthritis and juvenile rheumatoid arthritis), atherosclerosis, cancer and metastatic cancer (including, for example, breast cancer, triple negative breast cancer, lung cancer, melanoma, colon cancer, renal cell carcinoma, prostate cancer, cancer of the cervix, bladder cancer, rectal cancer, esophageal cancer, liver cancer, mouth and throat cancer, multiple myeloma, ovarian cancer, stomach cancer, pancreatic cancer and retinal blastoma cancers), cancer therapy-related disability or cancer therapy side effects, hypertension, glaucoma, osteoporosis, sarcopenia, cachexia, stroke, myocardial infarction, atrial fibrillation, transplantation rejection, diabetes mellitus—Type I, diabetes mellitus—Type II, radiation exposure, HIV treatment side effects, chemical weapons exposure, poisoning, inflammation, nephropathy, Lewy body dementia, prion disease (including bovine spongiform encephalopathy, Creutzfeldt-Jakob disease, scrapie, chronic wasting disease, kuru and fatal familial insomnia), lordokyphosis, auto-immune disorders, loss of adipose tissue, psoriasis, Crohn's disease, asthma, the physiological effects of aging (including “cosmetic” effects, such as wrinkling, age spots, hair loss, reduction in subcutaneous adipose tissue and thinning of the skin), idiopathic myopathy (including, for example, idiopathic inflammatory myopathy, idiopathic inflammatory myositis, polymyositis, dermatomyositis, sporadic inclusion body myositis and juvenile myositis), multiple sclerosis, neuromyelitis optica (NMO, Devic's disease or Devic's syndrome), epilepsy and adrenoleukodystrophy (ALD, X-linked adrenoleukodystrophy, X-ALD, cerebral ALD or cALD).

A particularly preferred treatment group includes subjects who have been diagnosed with an AGE disorder affecting a restricted site of the body. Examples of AGE disorders that affect a restricted site of the body include brain tumors (including gliomas such as anaplastic astrocytoma, glioblastoma multiforme, oligodendroglioma and diffuse intrinsic pontine glioma, meningiomas, pituitary adenomas and nerve sheath tumors), meningitis, brain/cerebral abscess, late-stage neurological trypanosomiasis (sleeping sickness), cerebral edema, prion diseases, encephalitis, rabies, arthritis/osteoarthritis, degenerative joint disease, synovitis, bursitis, prostate cancer, eye cancer and eye disorders.

The Present Application includes 66 nucleotide and amino acid sequences in the Sequence Listing filed herewith. Variants of the nucleotide and amino acid sequences are possible. Known variants include substitutions, deletions and additions to the sequences shown in SEQ ID NO: 4, 16 and 20. In SEQ ID NO: 4, the arginine (Arg or R) residue at position 128 may optionally be omitted. In SEQ ID NO: 16, the alanine residue at position 123 may optionally be replaced with a serine residue, and/or the tyrosine residue at position 124 may optionally be replaced with a phenylalanine residue. SEQ ID NO: 20 may optionally include the same substitutions as SEQ ID NO: 16 at positions 123 and 124. In addition, SEQ ID NO: 20 may optionally contain one additional lysine residue after the terminal valine residue.

### EXAMPLES

### Example 1: In Vivo Study of the Administration of Anti-Glycation End-Product Antibody

To examine the effects of an anti-glycation end-product antibody, the antibody was administered to the aged CD1(1CR) mouse (Charles River Laboratories), twice daily by intravenous injection, once a week, for three weeks (Days 1, 8 and 15), followed by a 10 week treatment-free period. The test antibody was a commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, Minn.; catalog no. MAB3247). A control reference of physiological saline was used in the control animals.

Mice referred to as “young” were 8 weeks old, while mice referred to as “old” were 88 weeks (±2 days) old. No adverse events were noted from the administration of the antibody. The different groups of animals used in the study are shown in Table 1.

TABLE 1
The different groups of animals used in the study

| | | | | Number of Animals | |
|---|---|---|---|---|---|
| | | | | | Treatment- |
| Group | Test | | Dose Level | Main Study | Free |
| No. | Material | Mice | (pg/gm/BID/week) | Females | Females |
| 1 | Saline | young | 0 | 20 | — |
| 2 | Saline | old | 0 | 20 | 20 |
| 3 | Antibody | old | 2.5 | 20 | 20 |
| 4 | None | old | 0 | 20 | pre |
| 5 | Antibody | old | 5.0 | 20 | 20 |

— = Not Applicable, Pre = Subset of animals euthanized prior to treatment start for collection of adipose tissue.

P16^{INK4a} mRNA, a marker for senescent cells, was quantified in adipose tissue of the groups by Real Time-qPCR. The results are shown in Table 2. In the table ΔΔCt=ΔCt mean control Group (2)—ΔCt mean experimental Group (1 or 3 or 5); Fold Expression=2^{-ΔΔCt}.

TABLE 2
P16^{INK4a} mRNA quantified in adipose tissue

| Calculation | Group 2 vs | | Group 2 vs | | Group 2 vs | |
|---|---|---|---|---|---|---|
| (unadjusted to | Group 1 | | Group 3 | | Group 5 | |
| Group 4: 5.59) | Group 2 | Group 1 | Group 2 | Group 3 | Group 2 | Group 5 |
| Mean ΔCt | 5.79 | 7.14 | 5.79 | 6.09 | 5.79 | 7.39 |
| ΔΔCt | −1.35 | | −0.30 | | −1.60 | |
| Fold | 2.65 | | 1.23 | | 3.03 | |
| Expression | | | | | | |

The table above indicates that untreated old mice (Control Group 2) express 2.55-fold more p16^{Ink4a} mRNA than the untreated young mice (Control Group 1), as expected. This was observed when comparing Group 2 untreated old mice euthanized at end of recovery Day 85 to Group 1 untreated young mice euthanized at end of treatment Day 22. When results from Group 2 untreated old mice were compared to results from Group 3 treated old mice euthanized Day 85, it was observed that p16^{Ink4a} mRNA was 1.23-fold higher in Group 2 than in Group 3. Therefore, the level of p16^{Ink4a} mRNA expression was lower when the old mice were treated with 2.5 μg/gram/BID/week of antibody.

When results from Group 2 (Control) untreated old mice were compared to results from Group 5 (5 μg/gram) treated old mice euthanized Day 22, it was observed that p16^{Ink4a} mRNA was 3.03-fold higher in Group 2 (controls) than in Group 5 (5 μg/gram). This comparison indicated that the Group 5 animals had lower levels of p16^{Ink4a} mRNA expression when they were treated with 5.0 μg/gram/BID/week, providing p16^{Ink4a} mRNA expression levels comparable to that of the young untreated mice (i.e. Group 1). Unlike Group 3 (2.5 μg/gram) mice that were euthanized at end of recovery Day 85, Group 5 mice were euthanized at end of treatment Day 22.

These results indicate the antibody administration resulted in the killing of senescent cells.

The mass of the gastrocnemius muscle was also measured, to determine the effect of antibody administration on sarcopenia. The results are provided in Table 3. The results indicate that administration of the antibody increased muscle mass as compared to controls, but only at the higher dosage of 5.0 μg/gm/BID/week.

TABLE 3
Effect of antibody administration on mass of the gastrocnemius muscle

| | | | Weight relative to |
|---|---|---|---|
| | Summary | Absolute weight of | body mass of |
| Group | Information | Gastrocnemius Muscle | Gastrocnemius Muscle |
| 1 | Mean | 0.3291 | 1.1037 |
| | SD | 0.0412 | 0.1473 |
| | N | 20 | 20 |
| 2 | Mean | 0.3304 | 0.7671 |
| | SD | 0.0371 | 0.1246 |
| | N | 20 | 20 |
| 3 | Mean | 0.3410 | 0.7706 |
| | SD | 0.0439 | 0.0971 |
| | N | 19 | 19 |
| 5 | Mean | 0.4074 | 0.9480 |
| | SD | 0.0508 | 0.2049 |
| | N | 9 | 9 |

These results demonstrate that administration of antibodies that bind to AGEs of a cell resulted in a reduction of cells expressing p16^{Ink4a}, a biomarker of senescence. The data show that reducing senescent cells leads directly to an increase in muscle mass in aged mice. These results indicate that the loss of muscle mass, a classic sign of sarcopenia, can be treated by administration of antibodies that bind to AGEs of a cell.

### Example 2: Affinity and Kinetics of Test Antibody

The affinity and kinetics of the test antibody used in Example 1 were analyzed using Nα,Nα-bis(carboxymethyl)-L-lysine trifluoroacetate salt (Sigma-Aldrich, St. Louis, Mo.) as a model substrate for an AGE-modified protein of a cell. Label-free interaction analysis was carried out on a BIACORE™ T200 (GE Healthcare, Pittsburgh, Pa.), using a Series S sensor chip CM5 (GE Healthcare, Pittsburgh, Pa.), with Fc1 set as blank, and Fc2 immobilized with the test antibody (molecular weigh of 150,000 Da). The running buffer was a HBS-EP buffer (10 mM HEPES, 150 mM NaCl, 3 mM EDTA and 0.05% P-20, pH of 7.4), at a temperature of 25° C. Software was BIACORE™ T200 evaluation software, version 2.0. A double reference (Fc2-1 and only buffer injection), was used in the analysis, and the data was fitted to a Langmuir 1:1 binding model.

TABLE 4
Experimental set-up of affinity and kinetics analysis
Association and dissociation

| Flow path | Fc1 and Fc2 |
|---|---|
| Flow rate (μl/min.) | 30 |
| Association time (s) | 300 |
| Dissociation time (s) | 300 |
| Sample concentration (μM) | 20 − 5 − 1.25 (x2) − 0.3125 − 0.078 − 0 |

A graph of the response versus time is illustrated in FIG. 1. The following values were determined from the analysis: kₐ (1/Ms)=1.857×10³⁻; k_{d} (1/s)=6.781×10⁻³; K_{D} (M)=3.651×10⁻⁶; Rₘₐₓ (RU)=19.52; and Chi²=0.114. Because the Chi² value of the fitting is less than 10% of Rₘₐₓ, the fit is reliable.

### Example 3: Construction and Production of Murine Anti-AGE IgG2b Antibody and Chimeric Anti-AGE IgG1 Antibody

Murine and chimeric human anti-AGE antibodies were prepared. The DNA sequence of murine anti-AGE antibody IgG2b heavy chain is shown in SEQ ID NO: 12. The DNA sequence of chimeric human anti-AGE antibody IgG1 heavy chain is shown in SEQ ID NO: 13. The DNA sequence of murine anti-AGE antibody kappa light chain is shown in SEQ ID NO: 14. The DNA sequence of chimeric human anti-AGE antibody kappa light chain is shown in SEQ ID NO: 15. The gene sequences were synthesized and cloned into high expression mammalian vectors. The sequences were codon optimized. Completed constructs were sequence confirmed before proceeding to transfection.

HEK293 cells were seeded in a shake flask one day before transfection, and were grown using serum-free chemically defined media. The DNA expression constructs were transiently transfected into 0.03 liters of suspension HEK293 cells. After 20 hours, cells were sampled to obtain the viabilities and viable cell counts, and titers were measured (OCTET® QKe, ForteBio). Additional readings were taken throughout the transient transfection production runs. The cultures were harvested on day 5, and an additional sample for each was measured for cell density, viability and titer.

The conditioned media for murine and chimeric anti-AGE antibodies were harvested and clarified from the transient transfection production runs by centrifugation and filtration. The supernatants were run over a Protein A column and eluted with a low pH buffer. Filtration using a 0.2 μm membrane filter was performed before aliquoting. After purification and filtration, the protein concentrations were calculated from the OD280 and the extinction coefficient. A summary of yields and aliquots is shown in Table 5:

TABLE 5
Yields and aliquots

| | Concentration | Volume | No. of | Total Yield |
|---|---|---|---|---|
| Protein | (mg/mL) | (mL) | vials | (mg) |
| Murine anti-AGE | 0.08 | 1.00 | 3 | 0.24 |
| Chimeric anti-AGE | 0.23 | 1.00 | 3 | 0.69 |

Antibody purity was evaluated by capillary electrophoresis sodium-dodecyl sulfate (CE-SDS) analysis using LabChip® GXII, (PerkinElmer).

### Example 4: Binding of Murine (Parental) and Chimeric Anti-AGE Antibodies

The binding of the murine (parental) and chimeric anti-AGE antibodies described in Example 3 was investigated by a direct binding ELISA. An anti-carboxymethyl lysine (CML) antibody (R&D Systems, MAB3247) was used as a control. CML was conjugated to KLH (CML-KLH) and both CML and CML-KLH were coated overnight onto an ELISA plate. HRP-goat anti-mouse Fc was used to detect the control and murine (parental) anti-AGE antibodies. HRP-goat anti-human Fc was used to detect the chimeric anti-AGE antibody.

The antigens were diluted to 1 μg/mL in 1× phosphate buffer at pH 6.5. A 96-well microtiter ELISA plate was coated with 100 μL/well of the diluted antigen and let sit at 4° C. overnight. The plate was blocked with 1×PBS, 2.5% BSA and allowed to sit for 1-2 hours the next morning at room temperature. The antibody samples were prepared in serial dilutions with 1×PBS, 1% BSA with the starting concentration of 50 μg/mL. Secondary antibodies were diluted 1:5,000. 100 μL of the antibody dilutions was applied to each well. The plate was incubated at room temperature for 0.5-1 hour on a microplate shaker. The plate was washed 3 times with 1×PBS. 100 μL/well diluted HRP-conjugated goat anti-human Fc secondary antibody was applied to the wells. The plate was incubated for 1 hour on a microplate shaker. The plate was then washed 3 times with 1×PBS. 100 μL HRP substrate TMB was added to each well to develop the plate. After 3-5 minutes elapsed, the reaction was terminated by adding 100 μL of 1N HCl. A second direct binding ELISA was performed with only CML coating. The absorbance at OD450 was read using a microplate reader.

The OD450 absorbance raw data for the CML and CML-KLH ELISA is shown in the plate map below. 48 of the 96 wells in the well plate were used. Blank wells in the plate map indicate unused wells.

Plate map of CML and CML-KLH ELISA:

| Conc. | | | | | | | |
|---|---|---|---|---|---|---|---|
| (μg/mL) | 1 | 2 | 3 | 4 | 5 | 6 | 7 |
| 50 | 0.462 | 0.092 | 0.42 | | 1.199 | 0.142 | 1.852 |
| 16.67 | 0.312 | 0.067 | 0.185 | | 0.31 | 0.13 | 0.383 |
| 5.56 | 0.165 | 0.063 | 0.123 | | 0.19 | 0.115 | 0.425 |
| 1.85 | 0.092 | 0.063 | 0.088 | | 0.146 | 0.099 | 0.414 |
| 0.62 | 0.083 | 0.072 | 0.066 | | 0.108 | 0.085 | 0.248 |
| 0.21 | 0.075 | 0.066 | 0.09 | | 0.096 | 0.096 | 0.12 |
| 0.07 | 0.086 | 0.086 | 0.082 | | 0.098 | 0.096 | 0.098 |
| 0 | 0.09 | 0.085 | 0.12 | | 0.111 | 0.083 | 0.582 |
| | R&D | Parental | Chimeric | | R&D | Parental | Chimeric |
| | Positive | Anti- | Anti- | | Positive | Anti- | Anti- |
| | Control | AGE | AGE | | Control | AGE | AGE |

| | |
|---|---|
| CML-KLH Coat | CML Coat |

The OD450 absorbance raw data for the CML-only ELISA is shown in the plate map below. 24 of the 96 wells in the well plate were used. Blank wells in the plate map indicate unused wells.

Plate map of CML-only ELISA:

| Conc. | | | | | | | |
|---|---|---|---|---|---|---|---|
| (μg/mL) | 1 | 2 | 3 | 4 | 5 | 6 | 7 |
| 50 | 1.913 | 0.165 | 0.992 | | | | |
| 16.66667 | 1.113 | 0.226 | 0.541 | | | | |
| 5.555556 | 0.549 | 0.166 | 0.356 | | | | |
| 1.851852 | 0.199 | 0.078 | 0.248 | | | | |
| 0.617284 | 0.128 | 0.103 | 0.159 | | | | |
| 0.205761 | 0.116 | 0.056 | 0.097 | | | | |
| 0.068587 | 0.073 | 0.055 | 0.071 | | | | |
| 0 | 0.053 | 0.057 | 0.06 | | | | |
| | R&D | Parental | Chimeric | | | | |
| | Positive | Anti- | Anti- | | | | |
| | Control | AGE | AGE | | | | |

The control and chimeric anti-AGE antibodies showed binding to both CML and CML-KLH. The murine (parental) anti-AGE antibody showed very weak to no binding to either CML or CML-KLH. Data from repeated ELISA confirms binding of the control and chimeric anti-AGE to CML. All buffer control showed negative signal.

### Example 5: Humanized Antibodies

Humanized antibodies were designed by creating multiple hybrid sequences that fuse select parts of the parental (mouse) antibody sequence with the human framework sequences. Acceptor frameworks were identified based on the overall sequence identity across the framework, matching interface position, similarly classed CDR canonical positions, and presence of N-glycosylation sites that would have to be removed. Three humanized light chains and three humanized heavy chains were designed based on two different heavy and light chain human acceptor frameworks. The amino acid sequences of the heavy chains are shown in SEQ ID NO: 29, 31 and 33, which are encoded by the DNA sequences shown in SEQ ID NO: 30, 32 and 34, respectively. The amino acid sequences of the light chains are shown in SEQ ID NO: 35, 37 and 39, which are encoded by the DNA sequences shown in SEQ ID NO: 36, 38 and 40, respectively. The humanized sequences were methodically analyzed by eye and computer modeling to isolate the sequences that would most likely retain antigen binding. The goal was to maximize the amount of human sequence in the final humanized antibodies while retaining the original antibody specificity. The light and heavy humanized chains could be combined to create nine variant fully humanized antibodies.

The three heavy chains and three light chains were analyzed to determine their humanness. Antibody humanness scores were calculated according to the method described in Gao, S. H., et al., “Monoclonal antibody humanness score and its applications”, BMC Biotechnology, 13:55 (Jul. 5, 2013). The humanness score represents how human-like an antibody variable region sequence looks. For heavy chains a score of 79 or above is indicative of looking human-like; for light chains a score of 86 or above is indicative of looking human-like. The humanness of the three heavy chains, three light chains, a parental (mouse) heavy chain and a parental (mouse) light chain are shown below in Table 6:

TABLE 6
Antibody humanness

| Antibody | Humanness (Framework + CDR) |
|---|---|
| Parental (mouse) heavy chain | 63.60 |
| Heavy chain 1 (SEQ ID NO: 29) | 82.20 |
| Heavy chain 2 (SEQ ID NO: 31) | 80.76 |
| Heavy chain 3 (SEQ ID NO: 33) | 81.10 |
| Parental (mouse) light chain | 77.87 |
| Light chain 1 (SEQ ID NO: 35) | 86.74 |
| Light chain 2 (SEQ ID NO: 37) | 86.04 |
| Light chain 3 (SEQ IN NO: 39) | 83.57 |

Full-length antibody genes were constructed by first synthesizing the variable region sequences. The sequences were optimized for expression in mammalian cells. These variable region sequences were then cloned into expression vectors that already contain human Fc domains; for the heavy chain, the IgG1 was used.

Small scale production of humanized antibodies was carried out by transfecting plasmids for the heavy and light chains into suspension HEK293 cells using chemically defined media in the absence of serum. Whole antibodies in the conditioned media were purified using MabSelect SuRe Protein A medium (GE Healthcare).

Nine humanized antibodies were produced from each combination of the three heavy chains having the amino acid sequences shown in SEQ ID NO: 29, 31 and 33 and three light chains having the amino acid sequences shown in SEQ ID NO: 35, 37 and 39. A comparative chimeric parental antibody was also prepared. The antibodies and their respective titers are shown below in Table 7:

TABLE 7
Antibody titers

| Antibody | Titer (mg/L) |
|---|---|
| Chimeric parental | 23.00 |
| SEQ ID NO: 29 + SEQ ID NO: 35 | 24.67 |
| SEQ ID NO: 29 + SEQ ID NO: 37 | 41.67 |
| SEQ ID NO: 29 + SEQ ID NO: 39 | 29.67 |
| SEQ ID NO: 31 + SEQ ID NO: 35 | 26.00 |
| SEQ ID NO: 31 + SEQ ID NO: 37 | 27.33 |
| SEQ ID NO: 31 + SEQ ID NO: 39 | 35.33 |
| SEQ ID NO: 33 + SEQ ID NO: 35 | 44.00 |
| SEQ ID NO: 33 + SEQ ID NO: 37 | 30.33 |
| SEQ ID NO: 33 + SEQ ID NO: 39 | 37.33 |

The binding of the humanized antibodies may be evaluated, for example, by dose-dependent binding ELISA or cell-based binding assay.

### Example 6: In Vivo Study of the Administration of a Carboxymethyl Lysine Monoclonal Antibody

The effect of a carboxymethyl lysine antibody on tumor growth, metastatic potential and cachexia was investigated. In vivo studies were carried out in mice using a murine breast cancer tumor model. Female BALB/c mice (BALB/cAnNCrl, Charles River) were eleven weeks old on Day 1 of the study.

4T1 murine breast tumor cells (ATCC CRL-2539) were cultured in RPMI 1640 medium containing 10% fetal bovine serum, 2 mM glutamine, 25 μg/mL gentamicin, 100 units/mL penicillin G Na and 100 μg/mL streptomycin sulfate. Tumor cells were maintained in tissue culture flasks in a humidified incubator at 37° C. in an atmosphere of 5% CO₂ and 95% air.

The cultured breast cancer cells were then implanted in the mice. 4T1 cells were harvested during log phase growth and re-suspended in phosphate buffered saline (PBS) at a concentration of 1×10⁶ cells/mL on the day of implant. Tumors were initiated by subcutaneously implanting 1×10⁵ 4 T1 cells (0.1 mL suspension) into the right flank of each test animal. Tumors were monitored as their volumes approached a target range of 80-120 mm³. Tumor volume was determined using the formula: tumor volume=(tumor width)²(tumor length)/2. Tumor weight was approximated using the assumption that 1 mm³ of tumor volume has a weight of 1 mg. Thirteen days after implantation, designated as Day 1 of the study, mice were sorted into four groups (n=15/group) with individual tumor volumes ranging from 108 to 126 mm³ and a group mean tumor volume of 112 mm³. The four treatment groups are shown in Table 8 below:

TABLE 8
Treatment groups

| Group | Description | Agent | Dosing (μg/g) |
|---|---|---|---|
| 1 | Control | phosphate buffered saline | N/A |
| | | (PBS) | |
| 2 | Low-dose | carboxymethyl lysine | 5 |
| | | monoclonal antibody | |
| 3 | High-dose | carboxymethyl lysine | 10 |
| | | monoclonal antibody | |
| 4 | Observation only | None | N/A |

An anti-carboxymethyl lysine monoclonal antibody was used as a therapeutic agent. 250 mg of carboxymethyl lysine monoclonal antibody was obtained from R&D Systems (Minneapolis, Minn.). Dosing solutions of the carboxymethyl lysine monoclonal antibody were prepared at 1 and 0.5 mg/mL in a vehicle (PBS) to provide the active dosages of 10 and 5 μg/g, respectively, in a dosing volume of 10 mL/kg. Dosing solutions were stored at 4° C. protected from light.

All treatments were administered intravenously (i.v.) twice daily for 21 days, except on Day 1 of the study where the mice were administered one dose. On Day 19 of the study, i.v. dosing was changed to intraperitoneal (i.p.) dosing for those animals that could not be dosed i.v. due to tail vein degradation. The dosing volume was 0.200 mL per 20 grams of body weight (10 mL/kg), and was scaled to the body weight of each individual animal.

The study continued for 23 days. Tumors were measured using calipers twice per week. Animals were weighed daily on Days 1-5, then twice per week until the completion of the study. Mice were also observed for any side effects. Acceptable toxicity was defined as a group mean body weight loss of less than 20% during the study and not more than 10% treatment-related deaths. Treatment efficacy was determined using data from the final day of the study (Day 23).

The ability of the anti-carboxymethyl lysine antibody to inhibit tumor growth was determined by comparing the median tumor volume (MTV) for Groups 1-3. Tumor volume was measured as described above. Percent tumor growth inhibition (% TGI) was defined as the difference between the MW of the control group (Group 1) and the MTV of the drug-treated group, expressed as a percentage of the MW of the control group. % TGI may be calculated according to the formula: % TGI=(1−MTV_{treated}/MTV_{control})×100.

The ability of the anti-carboxymethyl lysine antibody to inhibit cancer metastasis was determined by comparing lung cancer foci for Groups 1-3. Percent inhibition (% Inhibition) was defined as the difference between the mean count of metastatic foci of the control group and the mean count of metastatic foci of a drug-treated group, expressed as a percentage of the mean count of metastatic foci of the control group. % Inhibition may be calculated according to the following formula: % Inhibition=(1−Mean Count of Foci_{treated}/Mean Count of Foci_{control})×100.

The ability of the anti-carboxymethyl lysine antibody to inhibit cachexia was determined by comparing the weights of the lungs and gastrocnemius muscles for Groups 1-3. Tissue weights were also normalized to 100 g body weight.

Treatment efficacy was also evaluated by the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study.

Statistical analysis was carried out using Prism (GraphPad) for Windows 6.07. Statistical analyses of the differences between Day 23 mean tumor volumes (MTVs) of two groups were accomplished using the Mann-Whitney U test. Comparisons of metastatic foci were assessed by ANOVA-Dunnett. Normalized tissue weights were compared by ANOVA. Two-tailed statistical analyses were conducted at significance level P=0.05. Results were classified as statistically significant or not statistically significant.

The results of the study are shown below in Table 9:

TABLE 9
Results

| | | | | | | | Gastroc. | Lung |
|---|---|---|---|---|---|---|---|---|
| | | | | % | | | weight/ | weight/ |
| | MTV | % | Lung | Inhibi- | | | normalized | normalized |
| Group | (mm³) | TGI | foci | tion | PR | CR | (mg) | (mg) |
| 1 | 1800 | N/A | 70.4 | N/A | 0 | 0 | 353.4/19.68 | 2799.4/ |
| | | | | | | | | 292.98 |
| 2 | 1568 | 13% | 60.3 | 14% | 0 | 0 | 330.4/21.62 | 2388.9/ |
| | | | | | | | | 179.75 |
| 3 | 1688 | 6% | 49.0 | 30% | 0 | 0 | 398.6/24.91 | 2191.6/ |
| | | | | | | | | 214.90 |

All treatment regimens were acceptably tolerated with no treatment-related deaths. The only animal deaths were non-treatment-related deaths due to metastasis. The % TGI trended towards significance (P>0.05, Mann-Whitney) for the 5 μg/g (Group 2) and 10 μg/g treatment group (Group 3). The % Inhibition trended towards significance (P>0.05, ANOVA-Dunnett) for the 5 μg/g treatment group. The % Inhibition was statistically significant (P≤0.01, ANOVA-Dunnett) for the 10 μg/g treatment group. The ability of the carboxymethyl lysine antibody to treat cachexia trended towards significance (P>0.05, ANOVA) based on a comparison of the organ weights of the lung and gastrocnemius between treatment groups and the control group. The results indicate that administration of an anti-carboxymethyl lysine monoclonal antibody is able to reduce cancer metastases. This data provides additional evidence that in vivo administration of anti-AGE antibodies can provide therapeutic benefits safely and effectively.

### Example 7: Treatment of Glioma in a Canine Model

Dogs with spontaneous gliomas will be selected for treatment. Gliomas will be verified by magnetic resonance imaging (MRI) and evaluation of physical symptoms. The dogs will be divided into five treatment groups as shown in Table 10 below:

TABLE 10
Treatment groups

| Group | Description | Agent |
|---|---|---|
| 1 | Anti-AGE antibody | Anti-CML antibody |
| 2 | Anti-AGE antibody | Anti-CML antibody and low-frequency |
| | and ultrasound | low intensity pulsed ultrasound |
| 3 | Control antibody | Canine IgG1 antibody and low-frequency |
| | and ultrasound | low intensity pulsed ultrasound |
| 4 | Ultrasound | Low-frequency low intensity pulsed |
| | | ultrasound |
| 5 | Control | None |

The anti-AGE antibody is a carboxymethyllysine antibody. The anti-AGE antibody is a chimeric antibody, caninized antibody or canine antibody. The control antibody is a canine IgG1 antibody (Novus Biologicals). The antibodies are administered at a dose of 10 mg/kg. The antibodies are administered intravenously at day 0 and once weekly for three weeks.

The ultrasound is administered simultaneously with the antibodies. The ultrasound is administered at a frequency of 5.7 MHz, an intensity of 90 mW/cm² and a pulse repetition frequency (PRF) of 6.0 kHz. The ultrasound is administered by a probe positioned perpendicular to the temporal window of each dog's cranium.

At the end of the three-week administration regimen all groups are evaluated by MRI for the presence and amount of glioma tumor and for physical symptoms. The animals in group 2 (anti-AGE antibody and ultrasound) will have the least amount of glioma and the fewest physical symptoms, followed by the animals in group 1 (anti-AGE antibody), the animals in group 4 (ultrasound) and the animals in group 3 (control antibody and ultrasound). The animals in group 5 (control) will have no improvement in symptoms. The combination of ultrasound and anti-AGE antibody is able to access the restricted site of the brain and cross the blood-brain barrier to provide the most effective therapy.

### Example 8: Treatment of Degenerative Joint Disease (Osteoarthritis) in a Canine Model

Dogs with spontaneous degenerative joint disease (osteoarthritis) in the stifle joint (the canine equivalent to the human knee) will be selected for treatment. Degenerative joint disease will be verified by magnetic resonance imaging (MRI) and evaluation of physical symptoms. The dogs will be divided into five treatment groups as shown in Table 11 below:

TABLE 11
Treatment groups

| Group | Description | Agent |
|---|---|---|
| 1 | Anti-AGE antibody | Anti-CML antibody |
| 2 | Anti-AGE antibody | Anti-CML antibody and low-frequency |
| | and ultrasound | low intensity pulsed ultrasound |
| 3 | Control antibody | Canine IgG1 antibody and low-frequency |
| | and ultrasound | low intensity pulsed ultrasound |
| 4 | Ultrasound | Low-frequency low intensity pulsed |
| | | ultrasound |
| 5 | Control | None |

The anti-AGE antibody is a carboxymethyllysine antibody. The anti-AGE antibody is a chimeric antibody, caninized antibody or canine antibody. The control antibody is a canine IgG1 antibody (Novus Biologicals). The antibodies are administered at a dose of 10 mg/kg. The antibodies are administered intravenously at day 0 and once weekly for three weeks.

The ultrasound is administered simultaneously with the antibodies. The ultrasound is administered at a frequency of 5.7 MHz, an intensity of 90 mW/cm² and a pulse repetition frequency (PRF) of 6.0 kHz. The ultrasound is administered by a probe positioned on the affected stifle joint.

At the end of the three-week administration regimen all groups are evaluated by MRI for the presence and amount of degenerative joint disease and for physical symptoms. The animals in group 2 (anti-AGE antibody and ultrasound) will have the least amount of degenerative joint disease and the fewest physical symptoms, followed by the animals in group 1 (anti-AGE antibody), the animals in group 4 (ultrasound) and the animals in group 3 (control antibody and ultrasound). The animals in group 5 (control) will have no improvement in symptoms. The combination of ultrasound and anti-AGE antibody is able to access the restricted site of the joint to provide the most effective therapy.

### Example 9: In Vitro Administration of Antibodies and Ultrasound to Glioblastoma and Glioblastoma/Astrocytoma Cells

Cell lines of glioblastoma cells (CRL-1620) and glioblastoma/astrocytoma cells (HTB-15) were exposed to antibodies (AB912), followed by administration of ultrasound. AB912 binds to proliferating cell nuclear antigen (PCNA), which is a protein involved in the DNA replication process. Expression of PCNA is associated with proliferation or neoplastic transformation.

The antibody was applied to the cells at a final concentration of 0.2 mg/ml, 30 minutes prior to the ultrasound exposure. The cells were exposed to ultrasound for 1 hour, at a frequency of 1 Mhz at 0.4 w/cm² and a 50% pulse. The base of the dish was approximately 3 mm from the ultrasound head. After exposure, the control group and the 1 hour without AB912 group were seeded at 2×10⁵ cells/cm² and the 1 hour with AB912 group was seeded at 1.1×10⁵.

Approximately 90% of CRL-1620 cells bound with the antibody showed significant detachment from the culture dish. Only approximately 10% of the cells not bound with antibody detached from the culture plate. The samples were incubated at 37° C. and supplemented with 5% CO₂ and allowed to culture for approximately 64 hours. After the incubation period, the cell cultures were observed under a microscope and no reattachment was observed. Several large cell clusters were observed floating in the media. These cells were manually disrupted and no viable cells were found. The results are shown in Table 12 below:

TABLE 12
Treatment of CRL-1620 cells

| Group | Percentage of detached cells |
|---|---|
| Control | 10% |
| 1 hr ultrasound | 10% |
| 1 hr ultrasound and antibody | 90% |

The same antibody administration and ultrasound administration procedures described above were repeated with HTB-15 cells. Approximately 85% of cells bound with the antibody showed significant detachment from the culture dish. Only approximately 10% of the cells not bound with the antibody detached from the culture plate. After the 64-hour incubation period, the cell cultures were observed under a microscope and no reattachment was observed. The results are shown in Table 13 below:

TABLE 13
Treatment of HTB-15 cells

| Group | Percentage of detached cells |
|---|---|
| Control | 10% |
| 1 hr ultrasound | 10% |
| 1 hr ultrasound and antibody | 85% |

After 24 hours, the cultures of both cell lines that were bound with the antibody, but did not have ultrasound administered to them showed no visible signs of toxicity. The cultures exposed to ultrasound but were not bound with the antibody also showed no toxicity. The cells that were exposed to the antibody and ultrasound did exhibit toxicity.

These results are a proof-of-principle that the administration of antibodies and ultrasound together may be used to kill cells. The combination therapy selectively targeted and killed glioblastoma and glioblastoma/astrocytoma cells, two types of cancer found in the restricted site of the brain when present in vivo. The results demonstrate that the combination of antibody and ultrasound administration results in enhanced cell killing as compared to administration of ultrasound alone.

### Example 10—Immunofluorescent Staining of Glioma Cell Panel

Glioma cells from the ATCC glioma cell line panel were trypsinized in 0.25% trypsin EDTA for approximately five minutes, then washed and resuspended to approximately 10⁵ cells/mL. 100 μL aliquots of the suspension were transferred to a round bottom 96-well plate and centrifuged to remove the medium. Each cell line was resuspended to 200 μL of 0.1 μg/mL 4′,6-diamidino-2-phenylindole (DAPI) in phosphate-buffered saline (PBS) for 10 minutes, and then washed three times. The cells were then resuspended in 10 μg/mL of AB912-T2-17G6 antibody (a proliferating cell nuclear antigen (PCNA) antibody) in PBS and incubated at 2-8° C. for 30 minutes. The cells were rinsed three times in PBS then suspended in 10 μg/mL of anti-human IgG (H&L) conjugated with Texas Red (a secondary antibody). For each wash, cells were centrifuged at 200×g for five minutes and supernatant was aspirated from each well. Immunofluorescent detection was used to verify binding of the antibody to the glioma cells.

### Example 11—In Vitro Anti-AGE Antibody Binding Studies

In vitro experiments investigated the binding of the humanized monoclonal anti-advanced glycation end-product (anti-AGE) antibody referred to as SIWA 318H (produced by SIWA Therapeutics, Inc.) to glioma and pancreatic cancer cells. SIWA 318H is known to bind to carboxymethyllysine-modified proteins. Binding was verified by immunofluorescent staining as described in Example 10.

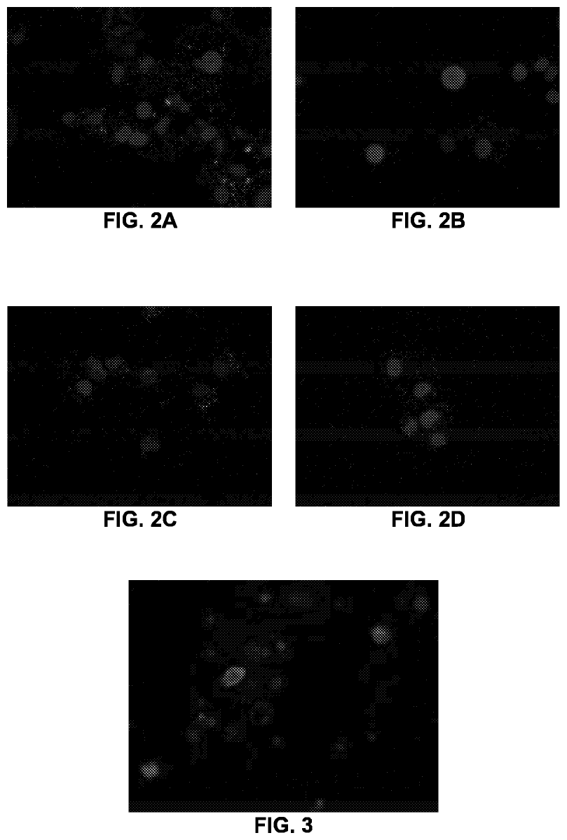

Pancreatic cancer cells from the human pancreatic cancer PANC-1 cell line were exposed to SIWA 318H. FIG. 2A-D illustrate the binding of SIWA 318H antibody to the pancreatic cancer cells.

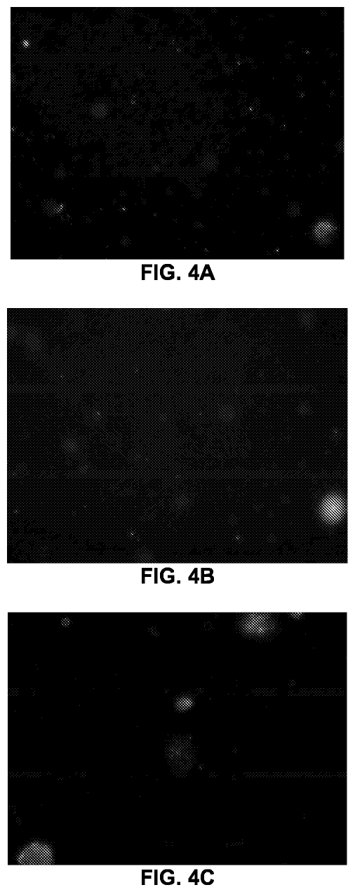

Glioma cells from the ATCC glioma cell line panel were exposed to SIWA 318H. SIWA 318H bound to all cell lines included in the panel (CRL-1620 human glioblastoma cells, HTB-12 human astrocytoma cells, HTB-148 human neuroglioma cells, HTB-15 human glioblastoma cells and HTB-14 human glioblastoma cells). Binding of SIWA 318H antibody to the glioma cells was confirmed by flow cytometry. FIG. 3 illustrates the binding of SIWA 318H antibody to glioma cells from the HTB-14 glioblastoma cell line. FIG. 4A-C illustrate the binding of SIWA 318H antibody to glioma cells from the HTB-15 glioblastoma cell line.

These results demonstrate that SIWA 318H, an anti-AGE antibody, binds to pancreatic cancer cells and glioma cells.

### REFERENCES

1. Myers, R. et al., “Ultrasound-mediated cavitation does not decrease the activity of small molecule, antibody or viral-based medicines”, International Journal of Nanomedicine, Vol. 13, pp. 337-349 (2018).
2. Liao, A-H. et al., “Enhanced therapeutic epidermal growth factor receptor (EGFR) antibody delivery via pulsed ultrasound with targeting microbubbles for glioma treatment”, Journal of Medical and Biological Engineering, Vol. 35, pp. 156-164 (2015).
3. Eguchi, K. et al., “Whole-brain low-intensity pulsed ultrasound therapy markedly improves cognitive dysfunctions in mouse models of dementia—crucial roles of endothelial nitric oxide synthase”, Brain Stimulation, pp. 1-15 (2018).
4. Lejbkowicz, F. et al., “Distinct sensitivity of normal and malignant cells to ultrasound in vitro”, Environmental Health Perspectives, Vol. 105, Supplement 6, pp. 1575-1578 (1997).
5. Ninomiya, K. et al., “Targeted sonodynamic therapy using protein-modified TiO₂ nanoparticles”, Ultrasonics Sonochemistry, Vol. 19, pp. 607-614 (2012).
6. Wood, A. K. W. et al., “A review of low-intensity ultrasound for cancer therapy”, Ultrasound in Medicine & Biology, Vol. 41, No. 4, pp. 905-928 (2015).
7. Berens, M. E. et al., “‘. . . those left behind.’ Biology and oncology of invasive glioma cells”, Neoplasia, Vol. 1, No. 3, pp. 208-219 (1999).
8. Lin, C-H. et al., “Advanced glycosylation end products induce nitric oxide synthase expression in C6 glioma cells involvement of a p38 MAP kinase-dependent mechanism”, Life Sciences, Vol. 69, pp. 2503-2515 (2001).
9. Masui, T. et al., “Low-intensity ultrasound enhances the anticancer activity of cetuximab in human head and neck cancer cells”, Experimental and Therapeutic Medicine, Vol. 5, pp. 11-16 (2013).
10. U.S. Pat. App. Pub. No. 2018/0036558.
11. U.S. Pat. No. 8,977,361.
12. U.S. Pat. App. Pub. No. 2017/0259086.
13. Neergaard, L., “Ultrasound jiggles open brain barrier, a step to better care”, available online at apnews.com/fdf368a847204e75bafa4bf700546d36, 1 page (2018).
14. Endo, S. et al., “Porphyrin derivatives-mediated sonodynamic therapy for malignant gliomas in vitro”, Ultrasound in Medicine and Biology, Vol. 41, No. 9, pp. 2458-2465 (2015).
15. Zhang, Z. et al., “Low frequency and intensity ultrasound induces apoptosis of brain glioma in rats mediated by caspase-3 Bcl-2, and survivin”, Brain Research, Vol. 1473, pp. 25-34 (2012).
16. Aviles Jr., F. et al., “Contact low-frequency ultrasound used to accelerate granulation tissue proliferation and rapid removal of nonviable tissue in colonized wounds: a case study”, Ostomy Wound Management, available online at www.ow-m.com/content/contact-low-frequency-ultrasound-used-accelerate-granulation-tissue-proliferation-and-rapid (2011).
17. Khaibullina, A. et al., “Pulsed high-intensity focused ultrasound enhances uptake of radiolabeled monoclonal antibody to human epidermoid tumor in nude mice”, Journal of Nuclear Medicine, Vol. 49, pp. 295-302 (2008).
18. Stoczynska-Fidelus, E., et al., “Spontaneous in vitro senescence of glioma cells confirmed by an antibody against IDH1^{R132H}”, Anticancer Research, Vol. 34, pp. 2859-2868 (2014).
19. Watson, K. D. et al., “Ultrasound increases nanoparticle delivery by reducing intratumoral pressure and increasing transport in epithelial and epithelial-mesenchymal transition tumors”, Cancer Research, Vol. 72, No. 6, pp. 1485-1493 (2012).
20. Chen, R. et al., “Ultrasound-accelerated immunoassay, as exemplified by enzyme immunoassay of choriogonadotropin”, Clinical Chemistry, Vol. 30, No. 9, pp. 1446-1451 (1984).
21. Wang, P. et al., “Membrane damage effect of continuous wave ultrasound on K562 human leukemia cells”, Journal of Ultrasound Medicine, Vol. 31, pp. 1977-1986 (2012).
22. Nisbet, R. M. et al., “Combined effects of scanning ultrasound and a tau-specific single chain antibody in a tau transgenic mouse model”, Brain, Vol. 140, pp. 1220-1230 (2017).
23. Zhao, B. et al., “Blood-brain barrier disruption induced by diagnostic ultrasound combined with microbubbles in mice”, Oncotarget, Vol. 9, No. 4, pp. 4897-4914 (2018).
24. Liu, H-L. et al., “Blood-brain barrier disruption with focused ultrasound enhances delivery of chemotherapeutic drugs for glioblastoma treatment”, Radiology, Vol. 255, No. 2, pp. 415-425 (2010).
25. Kinoshita, M. et al., “Noninvasive localized delivery of Herceptin to the mouse brain by MRI-guided focused ultrasound-induced blood-brain barrier disruption”, Proceedings of the National Academy of Sciences, Vol. 103, No. 31, pp. 11719-11723 (2006).
26. Liman, J. et al., “Transcranial ultrasound in adults and children with movement disorders”, Perspectives in Medicine, Vol. 1, pp. 349-352 (2012).
27. Purkayastha, S. et al., “Transcranial doppler ultrasound: technique and application”, Seminars in Neurology, Vol. 32, No. 4, pp. 411-420 (2012).
28. Sun, T. et al., “Closed-loop control of targeted ultrasound drug delivery across the blood-brain/tumor barriers in a rat glioma model”, Proceedings of the National Academy of Sciences, pp. 1-10 (2017).
29. Houston-Edwards, K., “Wave of the future?”, NOVA Next, available online at http://www.pbs.org/wgbh/nova/next/body/hifu/ (2016).
30. Allen, K. D. et al., “Evaluating intra-articular drug delivery for the treatment of osteoarthritis in a rat model”, Tissue Engineering Part B Reviews, Vol. 16, No. 1, pp. 81-92 (2010).
31. Udroiu, I., “Ultrasonic drug delivery in oncology”, JBUON, Vol. 20, No. 2, pp. 381-390 (2015).
32. Yu, T. et al., “Ultrasound: a chemotherapy sensitizer”, Technology in Cancer Research and Treatment, Vol. 5, No. 1, pp. 51-60 (2006).
33. Sawai, Y. et al., “Effects of low-intensity pulsed ultrasound on osteosarcoma and cancer cells”, Oncology Reports, Vol. 28, pp. 481-486 (2012).
34. Zhang, Z. et al., “Low intensity ultrasound promotes the sensitivity of rat brain glioma to doxorubicin by down-regulating the expressions of P-glucoprotein and multidrug resistance protein 1 in vitro and in vivo”, PLoS One, Vol. 8, Issue 8, e70685, pp. 1-13 (2013).
35. Takeuchi, R. et al., “Low-intensity pulsed ultrasound activates the phosphatidylinositol 3 kinase/Akt pathway and stimulates the growth of chondrocytes in three-dimensional cultures: a basic science study”, Arthritis Research & Therapy, Vol. 10, No. 4, pp. 1-11 (2008).
36. Rosenfeld, E. et al., “Positive and negative effects of diagnostic intensities of ultrasound on erythrocyte blood group markers”, Ultrasonics, Vol. 28, Issue 3, pp. 155-158 (1990).
37. Muhlfeld, J. et al., “Influence of ultrasonic waves and enzymes on antigenic properties of human erythrocytes. I. Ultrasonic waves”, Blut., Vol. 30, No. 5, pp. 349-352 (1975).
38. Cui, J. H. et al., “Effects of low-intensity ultrasound on chondrogenic differentiation of mesenchymal stem cells embedded in polyglycolic acid: an in vivo study”, Tissue Engineering, Vol. 12, No. 1 (2006).
39. Danno, D. et al., “Effects of ultrasound on apoptosis induced by anti-CD20 antibody in CD20-positive B lymphoma cells”, Ultrasonics Sonochemistry, Vol. 15, Issue 4, pp. 463-471 (2008).
40. Miller, D. et al., “Overview of therapeutic ultrasound applications and safety considerations”, Journal of Ultrasound in Medicine, Vol. 31, No. 4, pp. 623-634 (2012).

1. A method of killing AGE-modified cells, comprising:
applying ultrasound to a subject; and
administering to the subject a composition comprising an anti-AGE antibody.

2. (canceled)

3. A method of treating an AGE disorder, comprising:
applying ultrasound to a subject having an AGE disorder; and
administering to the subject a composition comprising an anti-AGE antibody.

4. A method of treating a subject with a glioma, comprising:
disrupting the blood-brain barrier of the subject; followed by
administering to the subject a composition comprising an anti-AGE antibody.

5. A method of treating a subject with osteoarthritis, comprising:
applying ultrasound to the affected joint; followed by
administering to the subject a composition comprising an anti-AGE antibody,
wherein the composition is administered intravenously.

6. The method of claim 3, wherein:
the anti-AGE antibody, is conjugated to a microbubble.

7-10. (canceled)

11. The method of claim 1, wherein the administering of the anti-AGE antibody occurs before the applying of ultrasound.

12. The method of claim 1, wherein the AGE-modified cells are in a restricted site.

13. The method of claim 1, wherein the restricted site is selected from the group consisting of the brain, a joint, the eyes, the testes, and the prostate.

14-18. (canceled)

19. The method of claim 1, wherein the subject is selected from the group consisting of humans, goats, sheep, pigs, cows, horses, camels, alpacas, dogs and cats.

20. The method of claim 1, wherein the subject is a human.

21. The method of claim 1, wherein the anti-AGE antibody is non-immunogenic to a species selected from the group consisting of humans, cats, dogs, horses, camels, alpaca, cattle, sheep, pigs, and goats.

22-23. (canceled)

24. The method of claim 1, wherein the anti-AGE antibody binds an AGE-modified cell comprising at least one protein or peptide that exhibits AGE modifications selected from the group consisting of FFI, pyrraline, AFGP, ALI, carboxymethyllysine, carboxyethyllysine and pentosidine.

25. The method of claim 1, wherein the anti-AGE antibody binds a carboxymethyllysine-modified protein or peptide.

26. (canceled)

27. The method of claim 1, wherein the AGE-modified cell is a senescent cell.

28-34. (canceled)

35. The method of claim 1, wherein the antibody is conjugated to an agent that causes the destruction of AGE-modified cells.

36-37. (canceled)

38. The method of claim 1, wherein the antibody is monoclonal.

39. The method of claim 1, wherein the antibody is substantially non-immunogenic to humans.

40. The method of claim 1, wherein the antibody has a rate of dissociation (k_{d}) of at most 9×10⁻³ sec⁻¹.

41-43. (canceled)

44. The method of claim 3, wherein the AGE disorder comprises at least one AGE disorder selected from the group consisting of brain cancer, brain tumors, gliomas, meningiomas, pituitary adenomas and nerve sheath tumors, meningitis, brain/cerebral abscess, late-stage neurological trypanosomiasis (sleeping sickness), cerebral edema, prion diseases, encephalitis, rabies, arthritis/osteoarthritis, degenerative joint disease, synovitis, bursitis, prostate cancer, eye cancer, and eye disorders.

45. The method of claim 1, wherein the ultrasound is applied at a frequency of 0.5-10 MHz,
an intensity of 0.05-5 W/cm², and
a pulse repetition frequency (PRF) of 0.05-10 kHz.

46-49. (canceled)
